(19) United States
(12) Patent Application Publication
(10) Pub. No.: US 2023/0372535 A1
(43) Pub. Date: Nov. 23, 2023

(54) SYNTHETIC NANOCARRIERS COMPRISING AN IMMUNOSUPPRESSANT IN COMBINATION WITH HIGH AFFINITY IL-2 RECEPTOR AGONISTS AND ANTI-IGM AGENTS

(71) Applicant: Selecta Biosciences, Inc., Watertown, MA (US)

(72) Inventor: Takashi Kei Kishimoto, Lexington, MA (US)

(73) Assignee: Selecta Biosciences, Inc., Watertown, MA (US)

(21) Appl. No.: 18/189,479

(22) Filed: Mar. 24, 2023

Related U.S. Application Data
(60) Provisional application No. 63/323,819, filed on Mar. 25, 2022.

(51) Int. Cl. A61K 47/69; C12N 15/86; A61K 45/06; B82Y 5/00

(52) U.S. Cl. CPC A61K 47/6931; C12N 15/86; A61K 45/06; B82Y 5/00

(57) ABSTRACT

Disclosed are methods and related compositions for administering a high affinity IL-2 receptor agonist, an anti-IgM agent and immunosuppressant (e.g., synthetic nanocarriers comprising immunosuppressant) in combination. The methods and compositions provided can be used for modulating an immune response to an antigen, such as by enhancing regulatory T cells, such as antigen-specific regulatory T cells.

### RELATED APPLICATIONS

This application claims the benefit of priority under 35 U.S.C. § 119(e) of U.S. Provisional Application No. 63/323,819, filed Mar. 25, 2022, the contents of which are incorporated herein by reference in their entirety.

### FIELD OF THE INVENTION

This invention relates, at least in part, to methods for administering a high affinity IL-2 receptor agonist and an anti-IgM agent in combination with immunosuppressant (e.g., synthetic nanocarriers comprising an immunosuppressant), and related compositions. The methods and compositions provided herein can be used for enhancing regulatory T cell (also referred to herein as Treg) induction, expansion and/or durability in a non-antigen specific manner and/or antigen-specific manner. The methods and compositions provided herein can be used for enhancing antigen-specific immune responses, such as antigen-specific immune responses of regulatory T cells. Thus, the methods, in some embodiments, can also include the administration of an antigen concomitantly with the high affinity IL-2 receptor agonist, anti-IgM agent, and synthetic nanocarriers. In some embodiments, the compositions, such as kits, provided herein can include an antigen, such as to which an antigen-specific tolerogenic immune response is desired. The methods and compositions provided herein can allow for a shift to tolerogenic immune response development, such as antigen-specific regulatory T cell production or development. The method and compositions provided herein can be used for subjects that would benefit from the enhancement of tolerogenic immune responses, such as antigen-specific regulatory T cell immune responses, or from the reduction of cytotoxic T cell activity.

### SUMMARY OF THE INVENTION

Undesired immune responses can be triggered by exposure to a particular antigen, such as a therapeutic macromolecule, an autoantigen or an allergen, or an antigen associated with an inflammatory disease, an autoimmune disease, organ or tissue rejection or graft versus host disease. Such undesired immune responses may be reduced through the use of immunosuppressant drugs. Conventional immunosuppressant drugs, however, are broad-acting. Additionally, in order to maintain immunosuppression, immunosuppressant drug therapy is generally a life-long proposition. Unfortunately, the use of broad-acting immunosuppressants can also be associated with a risk of severe side effects, such as tumors, infections, nephrotoxicity and metabolic disorders.

Accordingly, new tolerogenic therapies that can induce and expand regulatory T-cell production and development could be beneficial to suppress undesired immune reactions. High affinity IL-2 receptor agonists can, or be specifically engineered to, preferentially bind to and/or activate existing regulatory T-cells. Combination treatment with high affinity IL-2 receptor agonists, an anti-IgM agent, and immunosuppressant (e.g., synthetic nanocarriers comprising an immunosuppressant), and in some embodiments in the presence of or with administered antigen, can provide improved tolerogenic immune responses, for example, by expanding existing regulatory T cells and/or by inducing and/or expanding antigen-specific regulatory T cells. It has been surprisingly found that combination treatment with high affinity IL-2 receptor agonists, anti-IgM agents and immunosuppressant (e.g., synthetic nanocarriers comprising an immunosuppressant) can induce and/or expand existing regulatory T cells and/or induce and/or expand antigen-specific regulatory T cells.

In one aspect, a composition comprising immunosuppressant (e.g., synthetic nanocarriers comprising an immunosuppressant), an ant-IgM agent, and a high affinity IL-2 receptor agonist is provided. In some embodiments, the composition also comprises an antigen. In some embodiments, the antigen, anti-IgM agent, and high affinity IL-2 receptor agonist are each not co-formulated with the immunosuppressant (e.g., synthetic nanocarriers comprising the immunosuppessant). In one embodiment of any one of the compositions provided herein, the composition further comprises a pharmaceutically acceptable excipient.

One aspect of the disclosure provides a dosage form comprising any one of the compositions described herein.

In another aspect, a method comprising administering to a subject in need thereof a composition comprising immunosuppressant (e.g., synthetic nanocarriers comprising an immunosuppressant), an anti-IgM agent and a high affinity IL-2 receptor agonist is provided. In one embodiment, the method further comprises administering an antigen to the subject. In one embodiment, the administering of the synthetic nanocarriers, anti-IgM agent, and high affinity IL-2 receptor agonist is performed on a subject in which an antigen is present and against which a tolerogenic immune response is desired.

In one embodiment of any one of the methods provided herein, the immunosuppressant (e.g., synthetic nanocarriers comprising the immunosuppressant), the anti-IgM agent, and the high affinity IL-2 receptor agonist are administered concomitantly to the subject. In one embodiment of any one of the methods provided herein, the immunosuppressant (e.g., synthetic nanocarriers comprising the immunosuppressant), the anti-IgM agent, the high affinity IL-2 receptor agonist, and the antigen are administered concomitantly to the subject.

In one embodiment of any one of the methods or compositions provided herein, the antigen induces an undesired immune response in the subject. In one embodiment of any one of the methods or compositions provided herein, the antigen is one against which a tolerogenic immune response is desired.

In another embodiment of any one of the methods provided herein, the administration is in an amount effective to result in enhanced numbers (e.g., by percentage (or ratio)) of regulatory T cells, including antigen-specific regulatory T cells, and/or enhanced durability of regulatory T cells, including antigen-specific regulatory T cell activity.

In another embodiment of any one of the methods provided herein, the subject has or is at risk of having an inflammatory disease, an autoimmune disease, an allergy, organ or tissue rejection or graft versus host disease. In another embodiment of any one of the methods provided herein, the subject has undergone or will undergo transplantation. In another embodiment of any one of the methods provided herein, the subject has or is at risk of having an undesired immune response against an antigen that is being administered or will be administered to the subject.

In another embodiment of any one of the methods or compositions provided herein, the antigen is or is of any one of a therapeutic macromolecule, an autoantigen or an allergen, or an antigen associated with an inflammatory disease, an autoimmune disease, organ or tissue rejection or graft versus host disease. In another embodiment of any one of the methods or compositions provided herein, the therapeutic macromolecules are therapeutic proteins or therapeutic polynucleotides.

In another embodiment of any one of the methods or compositions provided herein, the therapeutic proteins are for protein replacement or protein supplementation therapy.

In another embodiment of any one of the methods or compositions provided herein, the therapeutic macromolecules comprise infusible or injectable therapeutic proteins, enzymes, enzyme cofactors, hormones, blood or blood coagulation factors, cytokines, interferons, growth factors, monoclonal antibodies, polyclonal antibodies or proteins associated with Pompe's disease.

In another embodiment of any one of the methods or compositions provided herein, the immunosuppressant comprises a statin, an mTOR inhibitor, a TGF-β signaling agent, a corticosteroid, an inhibitor of mitochondrial function, a P38 inhibitor, an NF-κB inhibitor, an adenosine receptor agonist, a prostaglandin E2 agonist, a phosphodiesterase 4 inhibitor, an HDAC inhibitor or a proteasome inhibitor. In another embodiment of any one of the methods or compositions provided herein, the mTOR inhibitor is rapamycin or a rapamycin analog.

In another embodiment of any one of the methods or compositions provided herein, the synthetic nanocarriers comprise lipid nanoparticles, polymeric nanoparticles, metallic nanoparticles, surfactant-based emulsions, dendrimers, buckyballs, nanowires, virus-like particles or peptide or protein particles. In another embodiment of any one of the methods or compositions provided herein, the synthetic nanocarriers comprise lipid nanoparticles. In another embodiment of any one of the methods or compositions provided herein, the synthetic nanocarriers comprise liposomes. In another embodiment of any one of the methods or compositions provided herein, the synthetic nanocarriers comprise metallic nanoparticles. In another embodiment of any one of the methods or compositions provided herein, the metallic nanoparticles comprise gold nanoparticles. In another embodiment of any one of the methods or compositions provided herein, the synthetic nanocarriers comprise polymeric nanoparticles.

In another embodiment of any one of the methods or compositions provided herein, the polymeric nanoparticles comprise a polymer that is a non-methoxy-terminated, pluronic polymer. In another embodiment of any one of the methods or compositions provided herein, the polymeric nanoparticles comprise a polyester, polyester coupled to a polyether, polyamino acid, polycarbonate, polyacetal, polyketal, polysaccharide, polyethyloxazoline or polyethyleneimine. In another embodiment of any one of the methods or compositions provided herein, the polyester comprises a poly(lactic acid), poly(glycolic acid), poly(lactic-co-glycolic acid) or polycaprolactone. In another embodiment of any one of the methods or compositions provided herein, the polymeric nanoparticles comprise a polyester and a polyester coupled to a polyether. In another embodiment of any one of the methods or compositions provided herein, the polyether comprises polyethylene glycol or polypropylene glycol.

In another embodiment of any one of the methods or compositions provided herein, the mean of a particle size distribution obtained using dynamic light scattering of the synthetic nanocarriers is a diameter greater than 100 nm. In another embodiment of any one of the methods or compositions provided herein, the diameter is greater than 110 nm, 120 nm, 130 nm, 140 nm or 150 nm. In another embodiment of any one of the methods or compositions provided herein, the diameter is greater than 200 nm. In another embodiment of any one of the methods or compositions provided herein, the diameter is greater than 250 nm. In another embodiment of any one of the methods or compositions provided herein, the diameter is greater than 300 nm. In another embodiment of any one of the methods or compositions provided herein, the diameter is less than 500 nm. In another embodiment of any one of the methods or compositions provided herein, the diameter is less than 450 nm. In another embodiment of any one of the methods or compositions provided herein, the diameter is less than 400 nm. In another embodiment of any one of the methods or compositions provided herein, the diameter is less than 350 nm. In some embodiments, the diameter is less than 300 nm. In some embodiments, the diameter is less than 250 nm. In some embodiments, the diameter is less than 200 nm. In some embodiments, the diameter is less than 150 nm.

In another embodiment of any one of the methods or compositions provided herein, an aspect ratio of the synthetic nanocarriers is greater than or equal to 1:1, 1:1.2, 1:1.5, 1:2, 1:3, 1:5, 1:7 or 1:10.

In another embodiment of any one of the methods or compositions provided herein, the load of the immunosuppressant on average across the synthetic nanocarriers is between 0.1% and 50% (weight/weight). In another embodiment of any one of the methods or compositions provided herein, the load of immunosuppressant on average across the synthetic nanocarriers is between 0.1% and 30% (weight/weight). In another embodiment of any one of the methods or compositions provided herein, the load of immunosuppressant on average across the synthetic nanocarriers is between 0.1% and 25% (weight/weight). In another embodiment of any one of the methods or compositions provided herein, the load of immunosuppressant is between 0.1% and 10% (weight/weight). In another embodiment of any one of the methods or compositions provided herein, the load of the immunosuppressant on average across the synthetic nanocarriers is between 1% and 50% (weight/weight). In another embodiment of any one of the methods or compositions provided herein, the load of immunosuppressant on average across the synthetic nanocarriers is between 1% and 30% (weight/weight). In another embodiment of any one of the methods or compositions provided herein, the load of immunosuppressant on average across the synthetic nanocarriers is between 1% and 25% (weight/weight). In another embodiment of any one of the methods or compositions provided herein, the load of immunosuppressant is between 1% and 10% (weight/weight). In another embodiment of any one of the methods or compositions provided herein, the load of the immunosuppressant on average across the synthetic nanocarriers is between 2% and 50% (weight/weight). In another embodiment of any one of the methods or compositions provided herein, the load of immunosuppressant on average across the synthetic nanocarriers is between 2% and 30% (weight/weight). In another embodiment of any one of the methods or compositions provided herein, the load of immunosuppressant on average across the synthetic nanocarriers is between 2% and 25% (weight/weight). In another embodiment of any one of the methods or compositions provided herein, the load of immunosuppressant is between 2% and 10% (weight/weight). In another embodiment of any one of the methods or compositions provided herein, the load of the immunosuppressant on average across the synthetic nanocarriers is between 4% and 50% (weight/weight). In another embodiment of any one of the methods or compositions provided herein, the load of immunosuppressant on average across the synthetic nanocarriers is between 4% and 30% (weight/weight). In another embodiment of any one of the methods or compositions provided herein, the load of immunosuppressant on average across the synthetic nanocarriers is between 4% and 25% (weight/weight). In another embodiment of any one of the methods or compositions provided herein, the load of immunosuppressant is between 4% and 10% (weight/weight). In another embodiment of any one of the methods or compositions provided herein, the load of the immunosuppressant on average across the synthetic nanocarriers is between 8% and 50% (weight/weight). In another embodiment of any one of the methods or compositions provided herein, the load of immunosuppressant on average across the synthetic nanocarriers is between 8% and 30% (weight/weight). In another embodiment of any one of the methods or compositions provided herein, the load of immunosuppressant on average across the synthetic nanocarriers is between 8% and 25% (weight/weight).

This invention also relates, at least in part, to repeated lower doses of a viral vector. The compositions and methods provided herein can be used to result in efficacious transgene expression while being dose sparing so as to provide, for example, reduced toxicity and/or manufacturing benefits. In addition, it has been surprisingly found that multiple lower doses of a viral vector in a dosing can result in comparable transgene expression as a higher dose of the viral vector, such as a single higher dose, such as a dose of 1e14 vector genomes/kg or 5e13 vector genomes/kg, with the methods and compositions provided herein.

In one embodiment of any one of the compositions or methods provided herein, the repeated lower doses of a viral vector of a dosing are each lower than a dose of the viral vector that would be administered to a subject without the administration (e.g., concomitant administration) of immunosuppressant (e.g., synthetic nanocarriers comprising an immunosuppressant, the anti-IgM agent and the IL-2 receptor agonist as provided herein to achieve the same or comparable level of transgene expression in a comparable subject or test subject. In one embodiment of any one of the compositions or methods provided herein, the repeated lower doses of a viral vector of a dosing is half or less than half a dose of the viral vector administered to a subject without the administration (e.g., concomitant administration) of immunosuppressant (e.g., synthetic nanocarriers comprising an immunosuppressant), the anti-IgM agent and the IL-2 receptor agonist as provided herein to achieve the same or comparable level of transgene expression.

In one embodiment of any one of the compositions or methods provided herein, the lower dose of a viral vector (e.g., 5E13 vg/kg) administered to a subject is given otherwise under the same conditions as a dose of the viral vector administered without the immunosuppressant (e.g., synthetic nanocarriers comprising an immunosuppressant), the anti-IgM agent, and the IL-2 receptor agonist as provided herein to achieve the same or comparable level of transgene expression to a comparable subject or test subject. In one embodiment of any one of the compositions or methods provided herein, the lower dose(s) of a viral vector (e.g., 5E13 vg/kg) of a dosing is concomitantly administered with immunosuppressant (e.g., synthetic nanocarriers comprising an immunosuppressant), the anti-IgM agent, and the IL-2 receptor agonist, such as when the synthetic nanocarriers are administered monthly, and/or the anti-IgM agent is administered at least biweekly and/or the IL-2 receptor agonist is administered monthly or every other month.

In one embodiment of any one of the compositions or methods provided herein, the lower dose is lower than a dose of the viral vector administered without concomitant administration of the immunosuppressant (e.g., synthetic nanocarriers that are attached to an immunosuppressant), the anti-IgM agent, and the IL-2 receptor agonist but results in comparable transgene expression and beneficial immune reduction as the dose of the viral vector administered without concomitant administration of the immunosuppressant (e.g., synthetic nanocarriers that are attached to an immunosuppressant), the anti-IgM agent, and the IL-2 receptor agonist.

In one embodiment of any one of the compositions or methods provided herein, a reduction in an undesired immune (eg., humoral) response to the viral vector and/or efficacious transgene or nucleic acid material expression and/or durable transgene or nucleic acid material expression and/or comparable transgene expression is the result of the dosing(s).

### BRIEF DESCRIPTION OF THE FIGURES

FIGS. 1A-1C show the effect of AAV8-SEAP, αBAFF, and IL-2 mutein injections alone and in combination on IgG and secreted embryonic alkaline phosphatase (SEAP) dynamics. FIG. 1A shows an exemplary treatment course used to inject mouse subjects. FIG. 1B shows the effect of AAV8-SEAP, αBAFF, and IL-2 mutein injections alone and in combination on SEAP activity. FIG. 1C shows IgG data.

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to “a therapeutic molecule” includes a mixture of two or more such therapeutic molecules or a plurality of such therapeutic molecules, reference to “an immunosuppressant” includes a mixture of two or more such materials or a plurality of such immunosuppressant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising” is inclusive and does not exclude additional, unrecited integers or method/process steps.

In embodiments of any one of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

### A. Introduction

As previously mentioned, current conventional immunosuppressants are broad-acting and generally result in an overall systemic downregulation of the immune system. It has been surprisingly found that beneficial effects can be achieved by practicing the methods described, or administering the compositions as provided herein. For example, treatment with synthetic nanocarriers comprising an immunosuppressant, an anti-IgM agent (e.g. anti-BAFF), and an IL-2 receptor agonist (e.g. IL-2 mutein) can suppress IgG production in response to treatment with a viral vector.

As described herein, combination treatment with an anti-IgM agent, high affinity IL-2 receptor agonists and immunosuppressant (e.g., synthetic nanocarriers comprising an immunosuppressant), and in some embodiments, in the presence of or with administered antigen, can provide improved antigen-specific immune responses. Such combinations can expand induced regulatory T cells, including antigen-specific Tregs. Accordingly, such methods and compositions can result in a decrease in undesired immune responses specific to a particular antigen (e.g., therapeutic macromolecule, an autoantigen or an allergen, or an antigen associated with an inflammatory disease, an autoimmune disease, organ or tissue rejection or graft versus host disease). The methods and compositions described herein may provide tolerance to or antigen-specific tolerogenic immune responses against a specific antigen.

The invention will now be described in more detail below.

### B. Definitions

“Administering” or “administration” or “administer” means providing a material to a subject in a manner that is pharmacologically useful. The term is intended to include “causing to be administered” in some embodiments. “Causing to be administered” means causing, urging, encouraging, aiding, inducing or directing, directly or indirectly, another party to administer the material.

“Amount effective” in the context of a composition or dosage form for administration to a subject refers to an amount of the composition or dosage form that produces one or more desired immune responses in the subject, for example, the reduction of an antibody response and/or the generation of a tolerogenic immune response, such as enhancement in the production or development of regulatory T cells, such as CD4+ regulatory T cells, such as those specific to a particular antigen, such as a therapeutic macromolecule, an autoantigen or an allergen, or an antigen associated with an inflammatory disease, an autoimmune disease, organ or tissue rejection or graft versus host disease. Therefore, in some embodiments, an amount effective is the amount of a composition or combination of compositions provided herein that produces one or more desired immune responses, such as an increase in the number or percentage (or ratio) of regulatory T cells, such as CD4+ regulatory T cells, that may or may not be antigen-specific and/or a decrease in an antibody response. The amount effective can be for in vitro or in vivo purposes. For in vivo purposes, the amount can be one that a clinician would believe may have a clinical benefit for a subject that may experience undesired immune responses to an antigen (e.g., a therapeutic macromolecule, an autoantigen or an allergen, or an antigen associated with an inflammatory disease, an autoimmune disease, organ or tissue rejection or graft versus host disease).

Amounts effective can involve reducing the level of an undesired immune response, although in some embodiments, it involves preventing an undesired immune response altogether. Amounts effective can also involve delaying the occurrence of an undesired immune response. An amount that is effective can also be an amount of a composition or combination of compositions provided herein that produces an increase in the production or development or durability of regulatory T cells (e.g., CD4+), such as antigen-specific regulatory T cells (e.g., CD4+). Specifically, the increase in the production or development can be an increase in the number of percentage (or ratio) of such cells. The increase can also be an increase in the activity of such cells. The increase can also be an increase in the durability of such cells. An amount effective can also be an amount that results in a desired therapeutic endpoint or a desired therapeutic result. Amounts effective, preferably, result in a tolerogenic immune response in a subject to an antigen. The achievement of any of the foregoing can be monitored by routine methods.

In some embodiments of any one of the compositions and methods provided, the amount effective is one in which the desired immune response persists in the subject for at least 1 week, at least 2 weeks, or at least 1 month. In other embodiments of any one of the compositions and methods provided, the amount effective is one which produces a measurable desired immune response, for example, a measurable decrease in an immune response (e.g., to a specific antigen), for at least 1 week, at least 2 weeks or at least 1 month.

Amounts effective will depend, of course, on the particular subject being treated; the severity of a condition, disease, or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason.

In general, doses of the IL-2 receptor agonist, anti-IgM agent, immunosuppressant and/or antigen refer to the amount of the IL-2 receptor agonist, anti-IgM agent, immunosuppressant and/or antigen. Alternatively, for the immunosuppressant and/or antigen, the dose can be administered based on the number of synthetic nanocarriers that provide the desired amount of immunosuppressant and/or antigen (e.g., the synthetic nanocarriers comprise the immunosuppressant and/or antigen). “Antigen-specific” refers to any immune response that results from the presence of the antigen, or portion thereof, or that generates molecules that specifically recognize or bind the antigen. For example, where the immune response is antigen-specific antibody production, antibodies are produced that specifically bind the antigen. As another example, the immune response is the production of regulatory T cells, which may be CD4+ regulatory T cells, that bind to an antigen-presenting cell (APC) presentable antigen when presented by an APC.

“Anti-BAFF agent” refers to any agent, small molecules, antibodies, peptides, or nucleic acids, that is known to reduce the production, or levels of, or activity of BAFF. In some embodiments, an anti-BAFF agent is an anti-BAFF antibody. Exemplary anti-BAFF agents include, but are not limited to, TACI-Ig and soluble BAFF receptor. “Anti-BAFF antibody” refers to any antibody that specifically binds to a BAFF polypeptide. For example, the anti-BAFF antibody may be a monoclonal antibody, such as Belimumab (Benlysta). In some instances, the anti-BAFF antibody can suppress the bioactivity of BAFF. Alternatively, or in addition, an anti-BAFF antibody may block the interaction between BAFF and its receptors, such as BAFF-R and BCMA (B cell maturation antigen). In some embodiments, a full intact antibody is used. In some embodiments, an antigen-binding fragment of the anti-BAFF antibody is instead used.

“Anti-IgM agent” refers to any agent, including but not limited to, small molecules, antibodies, peptides, or nucleic acids, that is known to reduce the production, or levels of, IgM, e.g., IgM antibodies. It will be appreciated by those of skill in the art that B cells generate antibodies. Thus, in some embodiments, an anti-IgM agent is any agent that is known to modulate or suppress B cell levels. In some embodiments, an anti-IgM agent is any agent that is known to modulate or suppress B cell maturation. In some embodiments, an anti-IgM agent is any agent that is known to modulate or suppress B cell activation. In some embodiments, an anti-IgM agent is any agent that is known to modulate or suppress T cell independent B cell activation.

Anti-IgM agents include, but are not limited to, IgM antagonist antibodies or antigen-binding fragments thereof that specifically bind to CD10, CD19, CD20, CD22, CD27, CD34, CD40, CD79a, CD79b, CD123, CD179b, FLT-3, ROR1, BR3, BAFF, or B7RP-1; IL21 modulating agents, e.g., IL-21 and IL-21 receptor antagonists; tyrosine kinase inhibitors, e.g., Syk inhibitors, BTK inhibitors, SRC protein tyrosine kinase inhibitors; PI3K inhibitors; PKC inhibitors; APRIL antagonists, e.g., TACI-Ig; mizoribine; tofacitinib; and tetracyclines.

An “antigen” is a natural or synthetic entity that is recognized as foreign by the antibodies or cells of the immune system and can trigger an immune response. Antigens can be in the form of peptides, proteins, polysaccharides or lipids (e.g., lipopolysaccharides). In some embodiments, antigens are generated in a subject as a result of normal cell metabolism. In some embodiments, an antigen is an autoantigen, a tumor antigen or a native antigen and can stimulate auto-antibodies (or immunoglobulins) in a subject. In some embodiments, antigens are involved in autoimmune disease pathogenesis. Non-limiting examples of antigens include therapeutic macromolecules such as those used for protein or enzyme replacement therapies, allergens such as those present in food products (e.g., peanuts, dairy, etc.) or other surrounding substances (e.g., pollen, latex, etc.), autoantigens in the case of autoimmune diseases, or other antigens associated with inflammatory diseases, autoimmune diseases, organ or tissue rejection or graft versus host disease. The antigen may be one to which a subject is exposed or is administered. The antigen may also be an endogenous antigen.

“Assessing an immune response” refers to any measurement or determination of the level, presence or absence, reduction, increase in, etc. of an immune response in vitro or in vivo. Such measurements or determinations may be performed on one or more samples obtained from a subject. Such assessing can be performed with any of the methods provided herein or otherwise known in the art. The assessing may be assessing the number or percentage of regulatory T cells, such as CD4+ regulatory T cells, such as those specific to a particular antigen, such as in a sample from a subject. In some embodiments, assessing may be assessing the production of immunoglobulins (e.g. IgG).

“Attach” or “Attached” or “Couple” or “Coupled” (and the like) means to chemically associate one entity (for example a moiety) with another. In some embodiments, the attaching is covalent, meaning that the attachment occurs in the context of the presence of a covalent bond between the two entities. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of attaching.

“Autoimmune disease” is a disease in which the immune system fails to recognize a subject's own organs, tissues or cells, and produces an immune response to attack those organs, tissues or cells as if they were foreign antigens. Autoimmune diseases are well known in the art; for example, as disclosed in The Encyclopedia of Autoimmune Diseases, Dana K. Cassell, Noel R. Rose, Infobase Publishing, 14 May 2014, incorporated by reference in its entirety as if fully disclosed herein.

“Average”, as used herein, refers to the arithmetic mean unless otherwise noted.

“Co-formulated” means that the indicated materials are processed so as to produce a filled and finished pharmaceutical dosage form wherein the materials are in intimate physical contact or are chemically attached covalently or non-covalently. As used herein, “not co-formulated” means that the indicated materials are not in intimate physical contact and are not chemically attached. In some embodiments, the IL-2 receptor agonist, anti-IgM agent, immunosuppressant (e.g., synthetic nanocarriers comprising the immunosuppressant) and/or antigen as described herein are not co-formulated prior to administration to a subject.

As used herein, the term “combination therapy” is intended to define therapies which comprise the use of a combination of two or more materials/agents. Thus, references to “combination therapy”, “combinations” and the use of materials/agents “in combination” in this application may refer to materials/agents that are administered as part of the same overall treatment regimen. As such, the posology of each of the two or more materials/agents may differ: each may be administered at the same time or at different times. It will therefore be appreciated that the materials/agents of the combination may be administered sequentially (e.g., before or after) or simultaneously, either in the same pharmaceutical formulation (i.e., together), or in different pharmaceutical formulations (i.e., separately). Simultaneously in the same formulation is as a unitary formulation whereas simultaneously in different pharmaceutical formulations is non-unitary. The posologies of each of the two or more materials/agents in a combination therapy may also differ with respect to the route of administration.

“Concomitantly” means administering two or more materials/agents to a subject in a manner that is correlated in time, preferably sufficiently correlated in time so as to provide a modulation in an immune response, and even more preferably the two or more materials/agents are administered in combination. In embodiments, concomitant administration may encompass administration of two or more materials/agents within a specified period of time, preferably within 1 month, more preferably within 1 week, still more preferably within 1 day, and even more preferably within 1 hour. In embodiments, the materials/agents may be repeatedly administered concomitantly; that is concomitant administration on more than one occasion.

“Determining” or “determine” means to ascertain a factual relationship. Determining may be accomplished in a number of ways, including but not limited to performing experiments, or making projections. For instance, a dose of an IL-2 receptor agonist, anti-IgM agent, immunosuppressant (e.g., synthetic nanocarriers comprising an immunosuppressant) and/or antigen may be determined by starting with a test dose and using known scaling techniques (such as allometric or isometric scaling) to determine the dose for administration. Such may also be used to determine a protocol as provided herein. In another embodiment, the dose may be determined by testing various doses in a subject, i.e., through direct experimentation based on experience and guiding data. In embodiments, “determining” or “determine” comprises “causing to be determined.” “Causing to be determined” means causing, urging, encouraging, aiding, inducing or directing or acting in coordination with an entity for the entity to ascertain a factual relationship; including directly or indirectly, or expressly or impliedly.

“Dosage form” means a pharmacologically and/or immunologically active material in a medium, carrier, vehicle, or device suitable for administration to a subject. Any one of the compositions or doses provided herein may be in a dosage form.

“Dose” refers to a specific quantity of a pharmacologically and/or immunologically active material for administration to a subject for a given time.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier.

“Enhancing the number or percentage of regulatory T cells” refers to increasing the number or percentage (or ratio) (of the total number of a type of cells) of said cells in a subject or subjects, as determined by taking samples from a subject or subjects and then assaying the samples using appropriate test methods. In some embodiments, by practicing the methods provided herein or following administration of the compositions described herein, the percentage of regulatory T cells, such as CD4+ regulatory T cells, such as those specific to a particular antigen, increases by at least 2-, 3-, 4-, 5-, or 6-fold or more.

CD4+ regulatory T cells can be characterized as CD4+CD25+FoxP3+ cells. The number or percentage of CD4+ regulatory T cells can be assessed by any method described herein or known in the art. For example, the CD4+ regulatory T cells in the peripheral blood of a subject can be quantified by obtaining a sample of peripheral blood from the subject, assessing the gene expression, protein presence, and/or localization of one or more molecules associated with CD4+ regulatory T cells, including without limitation CD25, FoxP3, CCR4, CCR8, CCR5, CTLA4, CD134, CD39, and/or GITR. Any of the foremetioned molecules can be assessed by transcriptional analysis, such as quantitative RT-PCR, northern blotting, microarray, fluorescence in situ hybridization, or RNAseq; proteins can be detected by western blotting, immunofluorescence microscopy, flow cytometry, or ELISA. Cell surface molecules such as CD25, CCR4, CCR8, CCR5, CTLA4, CD134, CD39 and/or GITR can be evaluated by methods such as flow cytometry, cell surface staining, immunofluorescence microscopy, ELISAs, etc. In some embodiments, CD4+ regulatory T cells are detected based on an anergic phenotype (e.g., lack of proliferation following TCR stimulation). In some embodiments, CD4+ regulatory T cells are identified based on resistance to activation-induced cell death or sensitivity to death induced by cytokine deprivation. In some embodiments, CD4+ regulatory T cells can be identified based on the methylation state of the gene encoding FoxP3; for example, in CD4+ regulatory T cells, a portion of the FoxP3 gene has been found to be demethylated, which can be detected by DNA methylation analysis such as by PCR or other DNA-based methods. CD4+ regulatory T cells can be further identified or quantified based on the production of immunosuppressive cytokines including IL-9, IL-10, or TGF-β. Antigen-specific CD4+ regulatory T cells can be identified and quantified by any method known in the art, for example, by stimulating cells ex vivo with an antigen-presenting cell loaded with the particular antigen and assessing activation of CD4+ regulatory T cells, or evaluating the T cell receptors of CD4+ regulatory T cells. The number or percentage (or ratio) of antigen-specific CD4+ regulatory T cells can be indirectly quantified by assessing one or more function or activity of activated CD4+ regulatory T cells following exposure to the antigen or a product containing the antigen.

“Generating” means causing an action, such as an immune response (e.g., a tolerogenic immune response) to occur, either directly oneself or indirectly.

A “high-affinity IL-2 receptor agonist” comprises a molecule that selectively binds to the high affinity receptor of interleukin-2 (IL-2) with high affinity and triggers a biological process at least similar in nature and intensity to the biological process that would be triggered by the binding of wild-type IL-2 to the high affinity IL-2 receptor. There are two major forms of the IL-2 receptor—a high affinity receptor comprised of an alpha (or CD25) chain, a beta chain and a gamma chain and a low (or moderate) affinity receptor comprised of just the beta and gamma chain. The high-affinity IL-2 receptor agonists as described herein selectively bind the high affinity receptor rather than the low affinity receptor. High-affinity IL-2 receptor agonists include but are not limited to wild-type IL-2, IL-2 muteins, IL-2 mimics, and fusion proteins of any of the foregoing (IL-2 fusion proteins). The wild-type IL-2 may be at a low dose or dosed in combination with specific monoclonal antibodies (mAbs), wherein the complex of the mAbs bound to IL-2 selectively binds the high affinity IL-2 receptor.

As used herein, “low-dose IL-2” refers to any dose of wild-type IL-2 a clinician would deem to be low. Such doses can be determined in one or more test subjects and applied to a subject in need of treatment. In some embodiments, the doses are seen in non-human test subjects and extrapolated to human subjects. In some embodiments of any one of the methods or compositions provided herein, a low dose of IL-2 is less than 5 million IU/m2, less than 4.5 million IU/m2, less than 4 IU/m2, or less than 3 IU/m2. In some embodiments of any one of the methods or compositions provided herein, a low dose of IL-2 is between 300,000 IU/m2 and 3 IU/m2. In some embodiments of any one of the methods or compositions provided herein, the low dose is an ultra-low dose. As used herein, an “ultra-low dose of IL-2” is any dose of wild-type IL-2 a clinician would deem to be an ultra-low dose. In some embodiments of any one of the methods or compositions provided herein, an ultra-low dose of IL-2 is less than 300,000 IU/m2. In some embodiments of any one of the methods or compositions provided herein, an ultra-low dose of IL-2 is less than 200,000 IU/m2. In some embodiments of any one of the methods or compositions provided herein, an ultra-low dose of IL-2 is between 50,000 IU/m2 and 200,000 IU/m2. In some embodiments, an ultra-low dose of IL-2 is 100,000 IU/m2.

In some embodiments, high affinity IL-2 receptor agonists are administered concomitantly with an immunosuppressant (e.g., synthetic nanocarriers comprising the immunosuppressant) and, optionally, a target antigen. Such administration can expand Tregs that are existing and/or specific to a target antigen. Without wishing to be bound by theory, the use of a high affinity IL-2 receptor agonist and the immunosuppressant (e.g., synthetic nanocarriers comprising the immunosuppressant) can synergistically induce and/or enhance the expansion of existing Tregs, which may include antigen-specific Tregs, and can provide for more durable immune tolerance, such as to a target antigen.

Any of the high affinity IL-2 receptor agonists provided herein can be in the form of a complex of mAbs bound thereto.

“Identifying a subject” is any action or set of actions that allows a clinician to recognize a subject as one who may benefit from the methods or compositions provided herein. Preferably, the identified subject is one who is in need of a tolerogenic immune response as provided herein, such as a subject in need of enhanced regulatory T cell production or development or durability, such as enhanced antigen-specific CD4+ regulatory T cell production or development or durability. The action or set of actions may be either directly oneself or indirectly. In one embodiment of any one of the methods provided herein, the method further comprises identifying a subject in need of a method or composition as provided herein.

“IgM antagonist antibodies” include, but are not limited to, antibodies that are known to reduce the production, or levels of, IgM, e.g., IgM antibodies. In some embodiments, an IgM antagonist antibody binds to and inhibits the activity of a protein or peptide involved in the production of, IgM, e.g., IgM antibodies, or in the modulation or stimulation immune pathway that leads to the production of, IgM, e.g., IgM antibodies.

In some embodiments, an IgM antagonist antibody is any antibody that is known to modulate B cell levels. In some embodiments, an IgM antagonist antibody is any antibody that is known to modulate B cell maturation. In some embodiments, an IgM antagonist antibody is any antibody that is known to modulate B cell activation. In some embodiments, an IgM antagonist antibody is any antibody that is known to modulate or suppress T cell independent B cell activation.

In some embodiments of any one of the methods, compositions or kits provided herein, an antigen-binding fragment of the antibody can be used in place of the antibody.

IgM antagonist antibodies or antigen-binding fragments thereof that specifically bind to CD10, CD19, CD20, CD22, CD27, CD34, CD40, CD79a, CD79b, CD123, CD179b, FLT-3, ROR1, BR3, BAFF, or B7RP-1 are examples of anti-IgM agents that can be used in any one of the methods, compositions or kits provided herein. Thus, such agents can also be antibodies or antigen-binding agents to B cell markers or other molecules that specifically bind such markers.

“IL-2 fusion proteins” refers to engineered proteins resulting from the fusion of an IL-2 molecules, such as wild-type IL-2, IL-2 muteins, IL-2 mimics, etc., or active portion thereof with one or more other peptide(s) or protein(s). Such other peptides or proteins may be antibodies or antigen-binding fragments thereof. The other peptides or proteins may also be an Fc portion of an IgG antibody, such as that may be used to extend the circulating half-life of the fusion protein. IL-2 fusion proteins may include IL-2 and anti-IL-2 antibodies or fusion proteins, IL-2-CD25 fusion proteins, etc.

“IL-2 mimics”, as used herein, refers to engineered proteins or functional fragments thereof designed to effect the same function(s) as IL-2 and selectively bind the high affinity IL-2 receptor. These proteins typically recapitulate the binding sites of IL-2 but differ from IL-2 in topology and/or amino acid sequence. An example of such IL-2 mimics is described in Silva, D A., Yu, S., Ulge, U. Y. et al. De novo design of potent and selective mimics of IL-2 and IL-15. Nature 565, 186-191 (2019). doi.org/10.1038/s41586-018-0830-7.

“IL-2 mutein” refers to a biologically active derivative of IL-2 that retains desired properties of IL-2 and selectively binds the high affinity IL-2 receptor. The term includes polypeptides having one or more amino acid-like molecules including but not limited to compounds comprising only amino and or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides.

Interleukin-2 (IL-2) is a cytokine that plays a pivotal role in T cell immunity and tolerance. During immune stimulation, IL-2 is an important cytokine that induces differentiation of CD4 and CD8 T cells into effector T cells following antigen-mediated activation. IL-2 also mediates differentiation of CD8 T cells into memory cells. However, IL-2 is also an important cytokine that mediates homeostasis and expansion of regulatory T cells (Tregs). Indeed, mice that are deficient in IL-2 develop lethal autoimmune syndrome. Effector T cells and Tregs express distinct receptors for IL-2. Tregs express a high affinity receptor for IL-2 comprised of three subunits, α (or CD25), β (or CD122) and γ (or CD132), while memory T cells express an intermediate affinity receptor comprised of only β and γ. While activated T cells can express CD25 after antigen stimulation, Tregs constitutively express high levels of CD25. Thus, Tregs are particularly sensitive to IL-2.

IL-2 can be engineered to produce IL-2 muteins. IL-2 muteins can be produced by introducing variations (such as mutations) into the amino acid chain of IL-2. Such mutations can be point mutations where one (or a few) amino acids are deleted, replaced (substituted) or added in the IL-2 chain. For example, it is possible to engineer IL-2 muteins to selectively bind to and activate T-regs. Such IL-2 muteins can have improved affinity for the IL-2 receptor a subunit and/or reduced affinity for the IL-2 receptor β and γ subunits, as compared to wild-type IL-2. IL-2 muteins can selectively promote the expansion of Treg cells and/or reduce agonism to effector T cells (Front Immunol. 2020 Apr. 28; 11:638. doi: 10.3389/fimmu.2020.00638, Sci Immunol. 2020 Aug. 14; 5(50):eaba5264. doi: 10.1126/sciimmunol.aba5264, Front Immunol. 2020 Jun. 5; 11:1106. doi: 10.3389/fimmu.2020.01106, Trends Immunol. 2015 December; 36(12):763-777. doi: 10.1016/j.it.2015.10.003, Semin Oncol. 2018 January; 45(1-2):95-104. doi: 10.1053/j.seminoncol.2018.04.001, US 2017/0037102 A1, J Immunol 2019 May 1; 202 (1 Supplement)68.20. doi). IL-2 muteins include, but are not limited to, PT101 (Pandion Therapeutics/Merck—engineered IL-2 mutein fused to and Fc protein backbone; J Immunol 2020 May 1; 204 (1 Supplement) 237.16), PT002 (Pandion Therapeutics/Merck—engineered IL-2 mutein with a MAdCAM tether for localization in the gut), N88D corresponding to a point mutation consisting of a substitution at amino acid position 88 of an Asparagine (N) residue with and Aspartic Acid (D) residue and the 2:1 stoichiometry IL-2 mutien-Fv fusion protein IgG-(IL-2N88D)2 (J. Autoimmun. 2018 Nov. 13; 95:1. doi.org/10.1016/j.jaut.2018.10.017), AMG 592 (Amgen—IL-2 mutein-Fc fusion protein), RG7835 (Roche—IL-2 mutein-Fc fusion protein). Other non-limiting examples of IL-2 muteins include, but are not limited to IL-2 with R38A, F42A, Y45A, and E62A mutations (J Immunol 2013 Jun. 15; 190(12):6230-8; doi: 10.4049/jimmunol.1201895), P85R IL-2 variant FSD13 (Cell Death Dis 9, 989 (2018). doi.org/10.1038/s41419-018-1047-2), no-alpha mutein (OncoImmunology 2020 June 2; 9:1; doi.org/10.1080/2162402X.2020.1770565), and other structurally modified IL-2 muteins (Front Immunol 2020 Jun. 5; 11(1106); doi.org/10.3389/fimmu.2020.01106, Protein Eng 2003 December; 16(12):1081-7; doi: 10.1093/protein/gzg111) as well as those of (J Exp Med 2020 Jan. 6; 217(1):e20191247; doi: 10.1084/jem.20191247, Nature 484, 529-533 (2012); doi.org/10.1038/nature10975, J Autoimmun 2015 January; 56:66-80; doi: 10.1016/j.jaut.2014.10.002).

“Immunosuppressant” means a compound that can cause an APC to have an immunosuppressive effect (e.g., tolerogenic effect) or a T or B cell to be suppressed. An immunosuppressive effect generally refers to the production or expression of cytokines or other factors by the APC that reduces, inhibits or prevents an undesired immune response or that promotes a desired immune response, such as a regulatory immune response (e.g., the production or development of regulatory T cells, such as CD4+ regulatory T cells). When the APC acquires an immunosuppressive function (under the immunosuppressive effect) on immune cells that recognize an antigen presented by this APC, the immunosuppressive effect is said to be specific to the presented antigen. Without being bound by any particular theory, it is thought that the immunosuppressive effect is a result of the immunosuppressant being delivered to the APC, preferably in the presence of an antigen. In one embodiment, the immunosuppressant is one that causes an APC to promote a regulatory phenotype in one or more immune effector cells. For example, the regulatory phenotype may be characterized by the inhibition of the production, induction, stimulation or recruitment of antigen-specific CD4+ T cells or B cells, the inhibition of the production of antigen-specific antibodies, the production, induction, stimulation or recruitment of Treg cells (e.g., CD4+CD25highFoxP3+ Treg cells), etc. This may be the result of the conversion of CD4+ T cells or B cells to a regulatory phenotype. This may also be the result of induction of FoxP3 in other immune cells, such as CD8+ T cells, macrophages and iNKT cells. In one embodiment, the immunosuppressant is one that affects the response of the APC after it processes an antigen. In another embodiment, the immunosuppressant is not one that interferes with the processing of the antigen. In a further embodiment, the immunosuppressant is not an apoptotic-signaling molecule. In another embodiment, the immunosuppressant is not a phospholipid.

Immunosuppressants include, but are not limited to, statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; histone deacetylase (HDAC) inhibitors, proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide. In embodiments, the immunosuppressant may comprise any of the agents provided herein.

The immunosuppressant can be a compound that directly provides the immunosuppressive effect on APCs or it can be a compound that provides the immunosuppressive effect indirectly (i.e., after being processed in some way after administration). Immunosuppressants, therefore, include prodrug forms of any of the compounds provided herein.

In embodiments of any one of the methods or compositions provided herein, the immunosuppressants provided herein are formulated with synthetic nanocarriers. In preferable embodiments, the immunosuppressant is an element that is in addition to the material that makes up the structure of the synthetic nanocarrier. For example, in one embodiment, where the synthetic nanocarrier is made up of one or more polymers, the immunosuppressant is a compound that is in addition and attached to (e.g., coupled) the one or more polymers. As another example, in one embodiment, where the synthetic nanocarrier is made up of one or more lipids, the immunosuppressant is again in addition and attached to the one or more lipids. In embodiments, such as where the material of the synthetic nanocarrier also results in an immunosuppressive effect, the immunosuppressant is an element present in addition to the material of the synthetic nanocarrier that results in an immunosuppressive effect.

Other exemplary immunosuppressants include, but are not limited, small molecule drugs, natural products, antibodies (e.g., antibodies against CD20, CD3, CD4), biologics-based drugs, carbohydrate-based drugs, nanoparticles, liposomes, RNAi, antisense nucleic acids, aptamers, methotrexate, NSAIDs; fingolimod; natalizumab; alemtuzumab; anti-CD3; tacrolimus (FK506), etc. Further immunosuppressants, are known to those of skill in the art, and the invention is not limited in this respect.

In embodiments of any one of the methods, compositions or kits provided herein, the immunosuppressant is in a form, such as a nanocrystalline form, whereby the form of the immunosuppressant itself is a particle or particle-like. In embodiments, such forms mimic a virus or other foreign pathogen. Many drugs have been nanonized and appropriate methods for producing such drug forms would be known to one of ordinary skill in the art. Drug nanocrystals, such as nanocrystalline rapamycin are known to those of ordinary skill in the art (Katteboinaa, et al. 2009, International Journal of PharmTech Resesarch; Vol. 1, No. 3; pp 682-694. As used herein a “drug nanocrystal” refers to a form of a drug (e.g., an immunosuppressant) that does not include a carrier or matrix material. In some embodiments, drug nanocrystals comprise 90%, 95%, 98% or 99% or more drug. Methods for producing drug nanocrystals include, without limitation, milling, high pressure homogenization, precipitation, spray drying, rapid expansion of supercritical solution (RESS), Nanoedge® technology (Baxter Healthcare), and Nanocrystal Technology™ (Elan Corporation). In some embodiments, a surfactant or a stabilizer may be used for steric or electrostatic stability of the drug nanocrystal. In some embodiments the nanocrystal or nanocrytalline form of an immunosuppressant may be used to increase the solubility, stability, and/or bioavailability of the immunosuppressant, particularly immunosuppressants that are insoluble or labile.

“Inflammatory disease” is a disease or condition characterized by abnormal inflammation, such as resulting from the immune system attacking a subject's own cells or tissues.

“Load”, when attached to a synthetic nanocarrier, is the amount of a molecule, such as an immunosuppressant and/or antigen, that can be attached to the synthetic nanocarrier based on the total dry recipe weight of materials in an entire synthetic nanocarrier (weight/weight). Generally, such a load is calculated as an average across a population of synthetic nanocarriers. In one embodiment, the load on average across the synthetic nanocarriers is between 0.0001% and 99%. In another embodiment, the load is between 0.1% and 50%. In another embodiment, the load is between 0.1% and 20%. In another embodiment, the load is between 0.1% and 25%. In a further embodiment, the load is between 0.1% and 10%. In still a further embodiment, the load is between 1% and 10%. In another embodiment, the load is between 1% and 25% or between 1% and 30%. In another embodiment, the load is between 2% and 25% or between 2% and 30%. In another embodiment, the load is between 4% and 25% or between 4% and 30%. In another embodiment, the load is between 8% and 25% or between 8% and 30%. In still a further embodiment, the load is between 7% and 20%. In yet another embodiment, the load is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 40%, or at least 50% on average across the population of synthetic nanocarriers. In yet a further embodiment, the load is 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20% on average across the population of synthetic nanocarriers. In some embodiments of the above embodiments, the load is no more than 25% on average across a population of synthetic nanocarriers. In embodiments, the load is calculated as otherwise known in the art. In one embodiment of any one of the foregoing load embodiments, the foregoing load embodiments refer to the load of immunosuppressant. In another embodiment of any one of the foregoing load embodiments, the foregoing load embodiments refer to the load of antigen. In one embodiment of such an embodiment the load of antigen (if also comprised in the synthetic nanocarriers) is between 1% and 10%.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., effective diameter) may be obtained, in some embodiments, by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicies of the sample. The effective diameter, or mean of the distribution, is then reported. Determining the effective sizes of high aspect ratio, or non-spheroidal, synthetic nanocarriers may require augmentative techniques, such as electron microscopy, to obtain more accurate measurements. “Dimension” or “size” or “diameter” of synthetic nanocarriers means the mean of a particle size distribution, for example, obtained using dynamic light scattering. In some embodiments, the mean of a particle size distribution obtained using dynamic light scattering of the synthetic nanocarriers is a diameter greater than 100 nm, 150 nm, 200 nm, 250 nm or 300 nm.

“Pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Protocol” means a pattern of administering to a subject and includes any dosing regimen of one or more substances to a subject. Protocols are made up of elements (or variables); thus a protocol comprises one or more elements. Such elements of the protocol can comprise dosing amounts, dosing frequency, routes of administration, dosing duration, dosing rates, interval between dosing, combinations of any of the foregoing, and the like. In some embodiments, such a protocol may be used to administer one or more compositions of the invention to one or more test subjects. Immune responses in these test subjects can then be assessed to determine whether or not the protocol was effective in generating a desired or desired level of an immune response or therapeutic effect. Any therapeutic and/or immunologic effect may be assessed. One or more of the elements of a protocol may have been previously demonstrated in test subjects, such as non-human subjects, and then translated into human protocols. For example, dosing amounts demonstrated in non-human subjects can be scaled as an element of a human protocol using established techniques such as alimetric scaling or other scaling methods. Whether or not a protocol had a desired effect can be determined using any of the methods provided herein or otherwise known in the art. For example, a sample may be obtained from a subject to which a composition provided herein has been administered according to a specific protocol in order to determine whether or not specific immune cells, cytokines, antibodies, etc. were reduced, generated, activated, etc. An exemplary protocol is one previously demonstrated to result in enhanced numbers or percentage (or ratio) of regulatory T cells, such as CD+ regulatory T cells with the methods or compositions provided herein. Useful methods for detecting the presence and/or number of immune cells include, but are not limited to, flow cytometric methods (e.g., FACS), ELISpot, proliferation responses, cytokine production, and immunohistochemistry methods. Antibodies and other binding agents for specific staining of immune cell markers, are commercially available. Such kits typically include staining reagents for antigens that allow for FACS-based detection, separation and/or quantitation of a desired cell population from a heterogeneous population of cells. In embodiments, a number of compositions as provided herein are administered to another subject using one or more or all or substantially all of the elements of which the protocol is comprised. In some embodiments, the protocol has been demonstrated to result in the development or production of existing or antigen-specific regulatory T cells, such as CD4+ regulatory T cells, with the methods or compositions as provided herein.

“Providing” means an action or set of actions that an individual performs that supply a needed item or set of items or methods for practicing of the present invention. The action or set of actions may be taken either directly oneself or indirectly.

“Providing a subject” is any action or set of actions that causes a clinician to come in contact with a subject and administer a composition provided herein thereto or to perform a method provided herein thereupon. Preferably, the subject is one who is in need of antigen-specific tolerance or enhanced production or development or durability of regulatory T cells as provided herein. The action or set of actions may be taken either directly oneself or indirectly. In one embodiment of any one of the methods provided herein, the method further comprises providing a subject.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like. In some embodiments, the subject has or is at risk of having an inflammatory disease, an autoimmune disease, an allergy, organ or tissue rejection or graft versus host disease. In other embodiments, the subject has undergone or will undergo transplantation. In further embodiments, the subject has or is at risk of having an undesired immune response against an antigen that is being administered or will be administered to the subject, such as a therapeutic macromolecule.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In some embodiments, synthetic nanocarriers do not comprise chitosan. In other embodiments, synthetic nanocarriers are not lipid-based nanoparticles. In further embodiments, synthetic nanocarriers do not comprise a phospholipid.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid attached virus-like particles disclosed in published US Patent Application 20060251677 to Bachmann et al., (9) the virus-like particles disclosed in WO2010047839A1 or WO2009106999A2, (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), (11) apoptotic cells, apoptotic bodies or the synthetic or semisynthetic mimics disclosed in U.S. Publication 2002/0086049, or (12) those of Look et al., Nanogel-based delivery of mycophenolic acid ameliorates systemic lupus erythematosus in mice” J. Clinical Investigation 123(4):1741-1749(2013). In some embodiments, synthetic nanocarriers may possess an aspect ratio greater than or equal to 1:1, 1:1.2, 1:1.5, 1:2, 1:3, 1:5, 1:7, or greater than 1:10.

Synthetic nanocarriers according to the invention that have a minimum dimension of equal to or less than about 100 nm, preferably equal to or less than 100 nm, in some embodiments, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, synthetic nanocarriers may possess an aspect ratio greater than or equal to 1:1, 1:1.2, 1:1.5, 1:2, 1:3, 1:5, 1:7, or 1:10.

A “therapeutic macromolecule” refers to any protein, carbohydrate, lipid or nucleic acid that may be administered to a subject and have a therapeutic effect. In some embodiments, administration of the therapeutic macromolecule to a subject may result in an undesired immune response. In some embodiments, the therapeutic macromolecule may be a therapeutic polynucleotide or therapeutic protein. In other embodiments, the therapeutic macromolecule comprises infusible or injectable therapeutic proteins, enzymes, enzyme cofactors, hormones, blood or blood coagulation factors, cytokines, interferons, growth factors, monoclonal antibodies, polyclonal antibodies or proteins associated with Pompe's disease.

“Therapeutic polynucleotide” means any polynucleotide or polynucleotide-based therapy that may be administered to a subject and have a therapeutic effect. Therapeutic polynucleotides may be produced in, on or by cells and also may be obtained using cell free or from fully synthetic in vitro methods. Subjects, therefore, include any subject that is in need of treatment with any of the foregoing. Such subject include those that will receive any of the foregoing. In an embodiment of any one of the methods or compositions provided herein, the therapeutic polynucleotide is a viral transfer vector.

“Therapeutic protein” means any protein or protein-based therapy that may be administered to a subject and have a therapeutic effect. Such therapies include protein replacement and protein supplementation therapies. Such therapies also include the administration of exogenous or foreign proteins, antibody therapies, and cell or cell-based therapies. Therapeutic proteins comprise, but are not limited to, infusible or injectable therapeutic proteins, enzymes, enzyme cofactors, hormones, blood clotting factors, cytokines, growth factors, monoclonal antibodies, antibody-drug conjugates, and polyclonal antibodies.

Therapeutic proteins may be produced in, on or by cells and may be obtained from such cells or administered in the form of such cells. In embodiments, the therapeutic protein is produced in, on or by mammalian cells, insect cells, yeast cells, bacteria cells, plant cells, transgenic animal cells, transgenic plant cells, etc. The therapeutic protein may be recombinantly produced in such cells. The therapeutic protein may be produced in, on or by a virally transformed cell. Subjects, therefore, include any subject that is in need of treatment with any of the foregoing. Such subjects include those that will receive any of the foregoing.

“Undesired immune response” refers to any undesired immune response, such as that that results from an antigen, promotes or exacerbates a disease, disorder or condition provided herein (or a symptom thereof), and/or is symptomatic of a disease, disorder or condition provided herein. Such immune responses generally have a negative impact on a subject's health or is symptomatic of a negative impact on a subject's health.

“Viral transfer vector” means a viral vector that has been adapted to deliver a nucleic acid, such as a transgene, as provided herein and includes such nucleic acid. “Viral vector” refers to all of the viral components of a viral transfer vector. Accordingly, “viral antigen” refers to an antigen of the viral components of the viral transfer vector, such as a capsid or coat protein, but not to the nucleic acid, such as a transgene, that it delivers, or any product it encodes. “Viral transfer vector antigen” refers to any antigen of the viral transfer vector including its viral components as well as delivered nucleic acid, such as a transgene, or any expression product thereof. The transgene may be a gene therapy transgene, a gene editing transgene, a gene expression modulating transgene or an exon skipping transgene. In some embodiments, the transgene is one that encodes a protein provided herein, such as a therapeutic protein, a DNA-binding protein or an endonuclease. In other embodiments, the transgene is one that encodes guide RNA, an antisense nucleic acid, snRNA, an RNAi molecule (e.g., dsRNAs or ssRNAs), miRNA, or triplex-forming oligonucleotides (TFOs), etc. Viral vectors can be based on, without limitation, retroviruses (e.g., murine retrovirus, avian retrovirus, Moloney murine leukemia virus (MoMuLV), Harvey murine sarcoma virus (HaMuSV), murine mammary tumor virus (MuMTV), gibbon ape leukemia virus (GaLV) and Rous Sarcoma Virus (RSV)), lentiviruses, herpes viruses, adenoviruses, adeno-associated viruses, alphaviruses, etc. Other examples are provided elsewhere herein or are known in the art. The viral vectors may be based on natural variants, strains, or serotypes of viruses, such as any one of those provided herein. The viral vectors may also be based on viruses selected through molecular evolution. The viral vectors may also be engineered vectors, recombinant vectors, mutant vectors, or hybrid vectors. In some embodiments, the viral vector is a “chimeric viral vector”. In such embodiments, this means that the viral vector is made up of viral components that are derived from more than one virus or viral vector.

### C. Compositions

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size or shape so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, synthetic nanocarriers can comprise one or more polymers. In some embodiments, the synthetic nanocarriers comprise one or more polymers that is a non-methoxy-terminated, pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated, pluronic polymers. In some embodiments, all of the polymers that make up the synthetic nanocarriers are non-methoxy-terminated, pluronic polymers. In some embodiments, the synthetic nanocarriers comprise one or more polymers that is a non-methoxy-terminated polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated polymers. In some embodiments, all of the polymers that make up the synthetic nanocarriers are non-methoxy-terminated polymers. In some embodiments, the synthetic nanocarriers comprise one or more polymers that do not comprise pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers do not comprise pluronic polymer. In some embodiments, all of the polymers that make up the synthetic nanocarriers do not comprise pluronic polymer. In some embodiments, such a polymer can be surrounded by a coating layer (e.g., liposome, lipid monolayer, micelle, etc.). In some embodiments, various elements of the synthetic nanocarriers can be attached to the polymer.

The immunosuppressants can be attached to the synthetic nanocarriers by any of a number of methods. Generally, the attaching can be a result of bonding between the immunosuppressants and the synthetic nanocarriers. This bonding can result in the immunosuppressants being attached to the surface of the synthetic nanocarriers and/or contained (encapsulated) within the synthetic nanocarriers. In some embodiments, however, the immunosuppressants are encapsulated by the synthetic nanocarriers as a result of the structure of the synthetic nanocarriers rather than bonding to the synthetic nanocarriers. In preferable embodiments, the synthetic nanocarrier comprises a polymer as provided herein, and the immunosuppressants are attached to the polymer.

When attaching occurs as a result of bonding between the immunosuppressants and synthetic nanocarriers, the attaching may occur via a coupling moiety. A coupling moiety can be any moiety through which an immunosuppressant is bonded to a synthetic nanocarrier. Such moieties include covalent bonds, such as an amide bond or ester bond, as well as separate molecules that bond (covalently or non-covalently) the immunosuppressant to the synthetic nanocarrier. Such molecules include linkers or polymers or a unit thereof. For example, the coupling moiety can comprise a charged polymer to which an immunosuppressant electrostatically binds. As another example, the coupling moiety can comprise a polymer or unit thereof to which it is covalently bonded.

In preferred embodiments, the synthetic nanocarriers comprise a polymer as provided herein. These synthetic nanocarriers can be completely polymeric or they can be a mix of polymers and other materials.

In some embodiments, the polymers of a synthetic nanocarrier associate to form a polymeric matrix. In some of these embodiments, a component, such as an immunosuppressant, can be covalently associated with one or more polymers of the polymeric matrix. In some embodiments, covalent association is mediated by a linker. In some embodiments, a component can be noncovalently associated with one or more polymers of the polymeric matrix. For example, in some embodiments, a component can be encapsulated within, surrounded by, and/or dispersed throughout a polymeric matrix. Alternatively or additionally, a component can be associated with one or more polymers of a polymeric matrix by hydrophobic interactions, charge interactions, van der Waals forces, etc. A wide variety of polymers and methods for forming polymeric matrices therefrom are known conventionally.

Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments, the polymer comprises a polyester, polycarbonate, polyamide, or polyether, or unit thereof. In other embodiments, the polymer comprises poly(ethylene glycol) (PEG), polypropylene glycol, poly(lactic acid), poly(glycolic acid), poly(lactic-co-glycolic acid), or a polycaprolactone, or unit thereof. In some embodiments, it is preferred that the polymer is biodegradable. Therefore, in these embodiments, it is preferred that if the polymer comprises a polyether, such as poly(ethylene glycol) or polypropylene glycol or unit thereof, the polymer comprises a block-co-polymer of a polyether and a biodegradable polymer such that the polymer is biodegradable. In other embodiments, the polymer does not solely comprise a polyether or unit thereof, such as poly(ethylene glycol) or polypropylene glycol or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g. attached) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly [α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids. Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids. In embodiments, the synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

Compositions according to the invention can comprise elements, such as immunosuppressants, in combination with pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, compositions, such as those comprising immunosuppressants, are suspended in sterile saline solution for injection together with a preservative.

In embodiments, when preparing synthetic nanocarriers as carriers, methods for attaching components to the synthetic nanocarriers may be useful. If the component is a small molecule it may be of advantage to attach the component to a polymer prior to the assembly of the synthetic nanocarriers. In embodiments, it may also be an advantage to prepare the synthetic nanocarriers with surface groups that are used to attach the component to the synthetic nanocarrier through the use of these surface groups rather than attaching the component to a polymer and then using this polymer conjugate in the construction of synthetic nanocarriers.

In certain embodiments, the attaching can be a covalent linker. In embodiments, immunosuppressants according to the invention can be covalently attached to the external surface via a 1,2,3-triazole linker formed by the 1,3-dipolar cycloaddition reaction of azido groups on the surface of the nanocarrier with immunosuppressant containing an alkyne group or by the 1,3-dipolar cycloaddition reaction of alkynes on the surface of the nanocarrier with immunosuppressants containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component such as an immunosuppressant with the carboxylic acid group of a second component such as the nanocarrier. The amide bond in the linker can be made using any of the conventional amide bond forming reactions with suitably protected amino acids and activated carboxylic acid such N-hydroxysuccinimide-activated ester.

A disulfide linker is made via the formation of a disulfide (S—S) bond between two sulfur atoms of the form, for instance, of R1-S—S—R2. A disulfide bond can be formed by thiol exchange of a component containing thiol/mercaptan group (—SH) with another activated thiol group on a polymer or nanocarrier or a nanocarrier containing thiol/mercaptan groups with a component containing activated thiol group.

A triazole linker, specifically a 1,2,3-triazole of the form

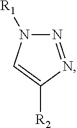

wherein R1 and R2 may be any chemical entities, is made by the 1,3-dipolar cycloaddition reaction of an azide attached to a first component such as the nanocarrier with a terminal alkyne attached to a second component such as the immunosuppressant. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The component is prepared with the presence of either an alkyne (if the polymer contains an azide) or an azide (if the polymer contains an alkyne) group. The component is then allowed to react with the nanocarrier via the 1,3-dipolar cycloaddition reaction with or without a catalyst which covalently attaches the component to the particle through the 1,4-disubstituted 1,2,3-triazole linker.

A thioether linker is made by the formation of a sulfur-carbon (thioether) bond in the form, for instance, of R1-S—R2. Thioether can be made by either alkylation of a thiol/mercaptan (—SH) group on one component with an alkylating group such as halide or epoxide on a second component. Thioether linkers can also be formed by Michael addition of a thiol/mercaptan group on one component to an electron-deficient alkene group on a second component containing a maleimide group or vinyl sulfone group as the Michael acceptor. In another way, thioether linkers can be prepared by the radical thiol-ene reaction of a thiol/mercaptan group on one component with an alkene group on a second component.

A hydrazone linker is made by the reaction of a hydrazide group on one component with an aldehyde/ketone group on the second component.

A hydrazide linker is formed by the reaction of a hydrazine group on one component with a carboxylic acid group on the second component. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component with an aldehyde or ketone group on the second component.

An urea or thiourea linker is prepared by the reaction of an amine group on one component with an isocyanate or thioisocyanate group on the second component.

An amidine linker is prepared by the reaction of an amine group on one component with an imidoester group on the second component.

An amine linker is made by the alkylation reaction of an amine group on one component with an alkylating group such as halide, epoxide, or sulfonate ester group on the second component. Alternatively, an amine linker can also be made by reductive amination of an amine group on one component with an aldehyde or ketone group on the second component with a suitable reducing reagent such as sodium cyanoborohydride or sodium triacetoxyborohydride.

A sulfonamide linker is made by the reaction of an amine group on one component with a sulfonyl halide (such as sulfonyl chloride) group on the second component.

A sulfone linker is made by Michael addition of a nucleophile to a vinyl sulfone. Either the vinyl sulfone or the nucleophile may be on the surface of the nanocarrier or attached to a component.

The component can also be conjugated to the nanocarrier via non-covalent conjugation methods. For example, a negative charged immunosuppressant can be conjugated to a positive charged nanocarrier through electrostatic adsorption. A component containing a metal ligand can also be conjugated to a nanocarrier containing a metal complex via a metal-ligand complex.

In embodiments, the component can be attached to a polymer, for example polylactic acid-block-polyethylene glycol, prior to the assembly of the synthetic nanocarrier or the synthetic nanocarrier can be formed with reactive or activatable groups on its surface. In the latter case, the component may be prepared with a group which is compatible with the attachment chemistry that is presented by the synthetic nanocarriers' surface. In other embodiments, a peptide component can be attached to VLPs or liposomes using a suitable linker. A linker is a compound or reagent that capable of coupling two molecules together. In an embodiment, the linker can be a homobifuntional or heterobifunctional reagent as described in Hermanson 2008. For example, an VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a peptide component containing an acid group via the other end of the ADH linker on nanocarrier to produce the corresponding VLP or liposome peptide conjugate.

For detailed descriptions of available conjugation methods, see Hermanson G T “Bioconjugate Techniques”, 2nd Edition Published by Academic Press, Inc., 2008. In addition to covalent attachment the component can be attached by adsorption to a pre-formed synthetic nanocarrier or it can be attached by encapsulation during the formation of the synthetic nanocarrier.

Any immunosuppressant as provided herein can be used in the methods or compositions provided and can be, in some embodiments, attached to, or comprised in, synthetic nanocarriers. Immunosuppressants include, but are not limited to, statins; mTOR inhibitors, such as rapamycin or a rapamycin analog; TGF-β signaling agents; TGF-β receptor agonists; histone deacetylase (HDAC) inhibitors; corticosteroids; inhibitors of mitochondrial function, such as rotenone; P38 inhibitors; NF-κβ inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cyclosporine A, siRNAs targeting cytokines or cytokine receptors and the like.

Examples of statins include atorvastatin (LIPITOR®, TORVAST®), cerivastatin, fluvastatin (LESCOL®, LESCOL® XL), lovastatin (MEVACOR®, ALTOCOR®, ALTOPREV®), mevastatin (COMPACTIN®), pitavastatin (LIVALO®, PIAVA®), rosuvastatin (PRAVACHOL®, SELEKTINE®, LIPOSTAT®), rosuvastatin (CRESTOR®), and simvastatin (ZOCOR®, LIPEX®).

Examples of mTOR inhibitors include rapamycin and analogs thereof (e.g., CCL-779, RAD001, AP23573, C20-methallylrapamycin (C20-Marap), C16-(S)-butylsulfonamidorapamycin (C16-BSrap), C16-(S)-3-methylindolerapamycin (C16-iRap) (Bayle et al. Chemistry & Biology 2006, 13:99-107)), AZD8055, BEZ235 (NVP-BEZ235), chrysophanic acid (chrysophanol), deforolimus (MK-8669), everolimus (RAD0001), KU-0063794, PI-103, PP242, temsirolimus, and WYE-354 (available from Selleck, Houston, TX, USA).

Examples of TGF-β signaling agents include TGF-β ligands (e.g., activin A, GDF1, GDF11, bone morphogenic proteins, nodal, TGF-βs) and their receptors (e.g., ACVR1B, ACVR1C, ACVR2A, ACVR2B, BMPR2, BMPR1A, BMPR1B, TGFβRI, TGFβRII), R-SMADS/co-SMADS (e.g., SMAD1, SMAD2, SMAD3, SMAD4, SMAD5, SMAD8), and ligand inhibitors (e.g, follistatin, noggin, chordin, DAN, lefty, LTBP1, THBS1, Decorin).

Examples of inhibitors of mitochondrial function include atractyloside (dipotassium salt), bongkrekic acid (triammonium salt), carbonyl cyanide m-chlorophenylhydrazone, carboxyatractyloside (e.g., from Atractylis gummifera), CGP-37157, (−)-Deguelin (e.g., from Mundulea sericea), F16, hexokinase II VDAC binding domain peptide, oligomycin, rotenone, Ru360, SFK1, and valinomycin (e.g., from Streptomyces fulvissimus) (EMD4Biosciences, USA).

Examples of P38 inhibitors include SB-203580 (4-(4-Fluorophenyl)-2-(4-methylsulfinylphenyl)-5-(4-pyridyl)1H-imidazole), SB-239063 (trans-1-(4hydroxycyclohexyl)-4-(fluorophenyl)-5-(2-methoxy-pyrimidin-4-yl) imidazole), SB-220025 (5-(2amino-4-pyrimidinyl)-4-(4-fluorophenyl)-1-(4-piperidinyl)imidazole)), and ARRY-797.

Examples of NF (e.g., NK-κβ) inhibitors include IFRD1, 2-(1,8-naphthyridin-2-yl)-Phenol, 5-aminosalicylic acid, BAY 11-7082, BAY 11-7085, CAPE (Caffeic Acid Phenethylester), diethylmaleate, IKK-2 Inhibitor IV, IMD 0354, lactacystin, MG-132 [Z-Leu-Leu-Leu-CHO], NFκB Activation Inhibitor III, NF-κB Activation Inhibitor II, JSH-23, parthenolide, Phenylarsine Oxide (PAO), PPM-18, pyrrolidinedithiocarbamic acid ammonium salt, QNZ, RO 106-9920, rocaglamide, rocaglamide AL, rocaglamide C, rocaglamide I, rocaglamide J, rocaglaol, (R)-MG-132, sodium salicylate, triptolide (PG490), and wedelolactone.

Examples of adenosine receptor agonists include CGS-21680 and ATL-146e.

Examples of prostaglandin E2 agonists include E-Prostanoid 2 and E-Prostanoid 4.

Examples of phosphodiesterase inhibitors (non-selective and selective inhibitors) include caffeine, aminophylline, IBMX (3-isobutyl-1-methylxanthine), paraxanthine, pentoxifylline, theobromine, theophylline, methylated xanthines, vinpocetine, EHNA (erythro-9-(2-hydroxy-3-nonyl)adenine), anagrelide, enoximone (PERFAN™), milrinone, levosimendon, mesembrine, ibudilast, piclamilast, luteolin, drotaverine, roflumilast (DAXAS™, DALIRESP™), sildenafil (REVATION®, VIAGRA®), tadalafil (ADCIRCA®, CIALIS®), vardenafil (LEVITRA®, STAXYN®), udenafil, avanafil, icariin, 4-methylpiperazine, and pyrazolo pyrimidin-7-1.

Examples of proteasome inhibitors include bortezomib, disulfiram, epigallocatechin-3-gallate, and salinosporamide A.

Examples of kinase inhibitors include bevacizumab, BIBW 2992, cetuximab (ERBITUX®), imatinib (GLEEVEC®), trastuzumab (HERCEPTIN®), gefitinib (IRESSA®), ranibizumab (LUCENTIS®), pegaptanib, sorafenib, dasatinib, sunitinib, erlotinib, nilotinib, lapatinib, panitumumab, vandetanib, E7080, pazopanib, and mubritinib.

Examples of glucocorticoids include hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (DOCA), and aldosterone.

Examples of retinoids include retinol, retinal, tretinoin (retinoic acid, RETIN-A®), isotretinoin (ACCUTANE®, AMNESTEEM®, CLARAVIS®, SOTRET®), alitretinoin (PANRETIN®), etretinate (TEGISON™) and its metabolite acitretin (SORIATANE®), tazarotene (TAZORAC®, AVAGE®, ZORAC®), bexarotene (TARGRETIN®), and adapalene (DIFFERIN®).

Examples of cytokine inhibitors include IL1ra, IL1 receptor antagonist, IGFBP, TNF-BF, uromodulin, Alpha-2-Macroglobulin, Cyclosporin A, Pentamidine, and Pentoxifylline (PENTOPAK®, PENTOXIL®, TRENTAL®).

Examples of peroxisome proliferator-activated receptor antagonists include GW9662, PPARγ antagonist III, G335, and T0070907 (EMD4Biosciences, USA).

Examples of peroxisome proliferator-activated receptor agonists include pioglitazone, ciglitazone, clofibrate, GW1929, GW7647, L-165,041, LY 171883, PPARγ activator, Fmoc-Leu, troglitazone, and WY-14643 (EMD4Biosciences, USA).

Examples of histone deacetylase inhibitors include hydroxamic acids (or hydroxamates) such as trichostatin A, cyclic tetrapeptides (such as trapoxin B) and depsipeptides, benzamides, electrophilic ketones, aliphatic acid compounds such as phenylbutyrate and valproic acid, hydroxamic acids such as vorinostat (SAHA), belinostat (PXD101), LAQ824, and panobinostat (LBH589), benzamides such as entinostat (MS-275), CI994, and mocetinostat (MGCD0103), nicotinamide, derivatives of NAD, dihydrocoumarin, naphthopyranone, and 2-hydroxynaphaldehydes.

Examples of calcineurin inhibitors include cyclosporine, pimecrolimus, voclosporin, and tacrolimus.

Examples of phosphatase inhibitors include BN82002 hydrochloride, CP-91149, calyculin A, cantharidic acid, cantharidin, cypermethrin, ethyl-3,4-dephostatin, fostriecin sodium salt, MAZ51, methyl-3,4-dephostatin, NSC 95397, norcantharidin, okadaic acid ammonium salt from prorocentrum concavum, okadaic acid, okadaic acid potassium salt, okadaic acid sodium salt, phenylarsine oxide, various phosphatase inhibitor cocktails, protein phosphatase 1C, protein phosphatase 2A inhibitor protein, protein phosphatase 2A1, protein phosphatase 2A2, and sodium orthovanadate.

In some embodiments of any one of the methods or compositions provided herein, the antigens, when also administered, can be attached to (e.g., encapsulated in) the synthetic nanocarriers to which the immunosuppressant is attached or to another population of synthetic nanocarriers that are not attached to the immunosuppressant. In other embodiments, the antigens are not attached to any synthetic nanocarriers. In some embodiments of either of these situations, the antigen may be delivered in the form of the antigen itself, or fragments or derivatives thereof. For example, therapeutic macromolecules may be delivered in the form of the therapeutic macromolecule itself, or fragments or derivatives thereof.

Therapeutic macromolecules can include therapeutic proteins or therapeutic polynucleotides. Therapeutic proteins include, but are not limited to, infusible therapeutic proteins, enzymes, enzyme cofactors, hormones, blood clotting factors, cytokines and interferons, growth factors, monoclonal antibodies, and polyclonal antibodies (e.g., that are administered to a subject as a replacement therapy), and proteins associated with Pompe's disease (e.g., acid glucosidase alfa, rhGAA (e.g., Myozyme and Lumizyme (Genzyme)). Therapeutic proteins also include proteins involved in the blood coagulation cascade. Therapeutic proteins include, but are not limited to, Factor VIII, Factor VII, Factor IX, Factor V, von Willebrand Factor, von Heldebrant Factor, tissue plasminogen activator, insulin, growth hormone, erythropoietin alfa, VEGF, thrombopoietin, lysozyme, antithrombin and the like. Therapeutic proteins also include adipokines, such as leptin and adiponectin.

Examples of therapeutic proteins used in enzyme replacement therapy of subjects having a lysosomal storage disorder include, but are not limited to, imiglucerase for the treatment of Gaucher's disease (e.g., CEREZYME™), a-galactosidase A (a-gal A) for the treatment of Fabry disease (e.g., agalsidase beta, FABRYZYME™), acid α-glucosidase (GAA) for the treatment of Pompe disease (e.g., acid glucosidase alfa, LUMIZYME™, MYOZYME™), arylsulfatase B for the treatment of Mucopolysaccharidoses (e.g., laronidase, ALDURAZYME™, idursulfase, ELAPRASE™, arylsulfatase B, NAGLAZYME™)), pegloticase (KRYSTEXXA) and pegsiticase.

Examples of enzymes include oxidoreductases, transferases, hydrolases, lyases, isomerases, asparaginases, uricases, glycosidases, asparaginases, uricases, proteases, nucleases, collagenases, hyaluronidases, heparinases, heparanases, lysins, and ligases.

Additional therapeutic proteins include, for example, engineered proteins, such as Fc fusion proteins, bispecific antibodies, multi-specific antibodies, nanobodies, antigen-binding proteins, antibody fragments, and protein conjugates, such as antibody drug conjugates.

Therapeutic polynucleotides include, but are not limited to nucleic acid aptamers such as Pegaptanib (Macugen, a pegylated anti-VEGF aptamer), antisense therapeutics such as antisense poly- or oligonucleotides (e.g., antiviral drug Fomivirsen, or Mipomersen, an antisense therapeutic that targets the messenger RNA for apolipoprotein B for reduction of cholesterol level); small interfering RNAs (siRNAs) (e.g., dicer substrate siRNA molecules (DsiRNAs) which are 25-30 base pair asymmetric double-stranded RNAs that mediate RNAi with extremely high potency); or modified messenger RNAs (mmRNAs) such as those disclosed in US Patent application 2013/0115272 to de Fougerolles et al. and in Published US Patent application 2012/0251618 to Schrum et al.

Additional therapeutic macromolecules useful in accordance with aspects of this invention will be apparent to those of skill in the art, and the invention is not limited in this respect.

In some embodiments, a component, such as an antigen, a high affinity IL-2 receptor agonist, anti-IgM agent or synthetic nanocarriers, may be isolated. Isolated refers to the element being separated from its native environment and present in sufficient quantities to permit its identification or use. This means, for example, the element may be (i) selectively produced by expression cloning or (ii) purified as by chromatography or electrophoresis. Isolated elements may be, but need not be, substantially pure. Because an isolated element may be admixed with a pharmaceutically acceptable excipient in a pharmaceutical preparation, the element may comprise only a small percentage by weight of the preparation. The element is nonetheless isolated in that it has been separated from the substances with which it may be associated in living systems, i.e., isolated from other lipids or proteins. Any of the elements provided herein may be isolated and included in the compositions or used in the methods in isolated form.

As used herein, “anti-IgM agents” are any agent that reduces the production of IgM, e.g., IgM antibodies. IgM antibodies are produced by B cells. While IgG antibodies are primarily produced in response to T cell-dependent activation of B cells, IgM antibodies are primarily produced in response to T cell-independent B cell activation, such as occurs in response to infection with viral vectors.

Anti-IgM agents include, but are not limited to, IgM antagonist antibodies or antigen-binding fragments thereof that specifically bind to CD10, CD19, CD20, CD22, CD27, CD34, CD40, CD79a, CD79b, CD123, CD179b, FLT-3, ROR1, BR3, BAFF, or B7RP-1; IL21 modulating agents, e.g., IL-21 and IL-21 receptor antagonists; tyrosine kinase inhibitors, e.g., Syk inhibitors, BTK inhibitors, SRC protein tyrosine kinase inhibitors; PI3K inhibitors; PKC inhibitors; APRIL antagonists, e.g., TACI-Ig; mizoribine; tofacitinib; and tetracyclines.

In some embodiments, the anti-IgM agent is an IgM antagonist antibody or antigen-binding fragment thereof. In some embodiments, the antibody targets a cell surface molecule on a B cell and binding of the antibody recruits the subject's immune system to attack and kill the B cell. In some embodiments, the antibody or antigen-binding fragment thereof specifically binds to CD10, CD19, CD20, CD22, CD27, CD34, CD40, CD79a, CD79b, CD123, CD179b, FLT-3, ROR1, BR3, BAFF, or B7RP-1.

In some embodiments, the antibody is an anti-CD10 antibody, e.g., an antibody that specifically binds CD10. Exemplary anti-CD10 antibodies include, but are not limited to, J5. In some embodiments, the antibody is an anti-CD27 antibody, e.g., an antibody that specifically binds CD27. CD27 is a member of the TNF receptor superfamily. In some embodiments, the antibody is an anti-CD34 antibody, e.g., an antibody that specifically binds CD34. In some embodiments, the antibody is an anti-CD79a antibody, e.g., an antibody that specifically binds CD79a. In some embodiments, the antibody is an anti-CD79b antibody, e.g., an antibody that specifically binds CD79b. Exemplary anti-CD79b antibodies include, but are not limited to, polatuzumab vedotin. In some embodiments, the antibody is an anti-CD123 antibody, e.g., an antibody that specifically binds CD123. Exemplary anti-CD123 antibodies include, but are not limited to, KHK2823 and CSL362. In some embodiments, the antibody is an anti-CD179b antibody, e.g., an antibody that specifically binds CD179b. In some embodiments, the antibody is an anti-FLT-3 antibody, e.g., an antibody that specifically binds FLT-3. Exemplary anti-FLT-3 antibodies include, but are not limited to, sorafenib and quizartinib. In some embodiments, the antibody is an anti-ROR1 antibody, e.g., an antibody that specifically binds ROR1. Exemplary anti-ROR1 antibodies include, but are not limited to, cirmtuzumab. In some embodiments, the antibody is an anti-BR3 antibody, e.g., an antibody that specifically binds BR3. In some embodiments, the antibody is an anti-B7RP-1 antibody, e.g., an antibody that specifically binds B7RP-1. Exemplary anti-B7RP-1 antibodies include, but are not limited to, prezalumab.

In some embodiments, the antibody is an anti-CD19 antibody, e.g., an antibody that specifically binds CD19. Exemplary anti-CD19 antibodies include, but are not limited to, MOR00208 (MorphoSysAG).

In some embodiments, the antibody is an anti-CD20 antibody, e.g., an antibody that specifically binds CD20. Exemplary anti-CD20 antibodies include, but are not limited to, rituximab, obinutuzumab, ocrelizumab, ofatumumab, iodine 131 tositumomab (Bexxar), ibritumomab, hyaluronidase/rituximab, and ibritumomab.

In some embodiments, the antibody is an anti-CD22 antibody, e.g., an antibody that specifically binds CD22. Exemplary anti-CD22 antibodies include, but are not limited to, epratuzumab and moxetumomab.

In some embodiments, the antibody is an anti-CD40 antibody, e.g., an antibody that specifically binds CD40. Exemplary anti-CD40 antibodies include, but are not limited to, ABBV-927 (Abbvie) and APX005M (Apexigen).

In some embodiments, the antibody is an anti-BAFF antibody or antigen-binding fragment thereof. BAFF, B cell activation factor (B lymphocyte stimulator), is an important cytokine for the generation and maintenance of B cells. BAFF has multiple receptors, which play a role in transmitting signals to different classes of B cells, such as BAFF-R, which is selective and important in early B-cell homeostasis and T-reg function and B-cell maturation antigen (BCMA), which is restricted to antibody-producing cells and is important for plasma cell longevity. Anti-BAFF antibodies, such as Belimumab, can include agents that specifically bind BAFF. Anti-BAFF antibodies may interfere with the interaction between BAFF and its receptors, such as BAFF-R and BCMA (B cell maturation antigen). Anti-BAFF antibodies are commercially available and one skilled in the art would be able to ascertain whether a certain agent is an anti-BAFF antibody. Any one of the anti-BAFF antibodies described herein or otherwise known, or antigen-binding fragments thereof, may be used in any one of the methods provided or be comprised in any one of the compositions or kits provided.

In some embodiments, the antibody or antigen-binding fragment thereof as described herein can bind and inhibit the activity of its target at least 50% (e.g., 60%, 70%, 80%, 90%, 95% or greater). The inhibitory activity of any of the antibodies or antigen-binding fragments thereof described herein can be determined by routine methods known in the art, for example, with an ELISA. Furthermore, binding affinity (or binding specificity) can be determined by a variety of methods including equilibrium dialysis, equilibrium binding, gel filtration, ELISA, surface plasmon resonance, or spectroscopy (e.g., using a fluorescence assay).

As used herein, “antibody” refers to a glycoprotein comprising at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “antigen-binding fragment” of an antibody refers to one or more portions of an antibody that retain the ability to bind specifically to an antigen. The antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, V and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference, as well as by other techniques known to those with skill in the art. The fragments can be screened for utility in the same manner as are intact antibodies.

In embodiments of any one of the methods or compositions or kits provided herein, the antibody or antigen-binding fragment thereof may be those produced by engineered sequences based on an antibody or antigen-binding fragment thereof.

Examples of antibodies described herein are commercially available and one skilled in the art would be able to ascertain whether a certain agent is a CD10, CD19, CD20, CD22, CD27, CD34, CD40, CD79a, CD79b, CD123, CD179b, FLT-3, ROR1, BR3, BAFF, or B7RP-1 antibody. Any one of the antibodies described herein or otherwise known, or antigen-binding fragments thereof, may be used in any one of the methods provided or be comprised in any one of the compositions or kits provided.

### D. Methods of Making and Using the Compositions and Related Methods

Synthetic nanocarriers may be prepared using a wide variety of methods known in the art. For example, synthetic nanocarriers can be formed by methods such as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

Various materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. No. 6,632,671 to Unger issued Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be attached to the synthetic nanocarriers and/or the composition of the polymer matrix.

If synthetic nanocarriers prepared by any of the above methods have a size range outside of the desired range, synthetic nanocarriers can be sized, for example, using a sieve.

Elements (i.e., components) of the synthetic nanocarriers may be attached to the overall synthetic nanocarrier, e.g., by one or more covalent bonds, or may be attached by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be attached to components directly or indirectly via non-covalent interactions. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such attachments may be arranged to be on an external surface or an internal surface of a synthetic nanocarrier. In embodiments, encapsulation and/or absorption is a form of attaching. In embodiments, the synthetic nanocarriers can be combined with an antigen by admixing in the same vehicle or delivery system.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention may comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, compositions are suspended in sterile saline solution for injection with a preservative.

It is to be understood that the compositions of the invention can be made in any suitable manner, and the invention is in no way limited to compositions that can be produced using the methods described herein. Selection of an appropriate method of manufacture may require attention to the properties of the particular moieties being associated.

In some embodiments, compositions are manufactured under sterile conditions or are terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection. In some embodiments, the compositions may be lyophilized and stored in suspension or as lyophilized powder depending on the formulation strategy for extended periods without losing activity.

Administration according to the present invention may be by a variety of routes, including but not limited to subcutaneous, intravenous, intraperitoneal, intramuscular, transmucosal, transdermal, transcutaneous or intradermal routes. In a preferred embodiment, administration is via a subcutaneous route of administration. The compositions referred to herein may be manufactured and prepared for administration, in some embodiments concomitant administration, using conventional methods.

The compositions of the invention can be administered in effective amounts, such as the effective amounts described elsewhere herein. Doses of dosage forms may contain varying amounts of anti-IgM agent, high affinity IL-2 receptor agonist, immunosuppressant (e.g., synthetic nanocarriers comprising an immunosuppressant) and/or antigen, according to the invention. The amount of anti-IgM agent, high affinity IL-2 receptor agonist, immunosuppressant (e.g., synthetic nanocarriers comprising an immunosuppressant) and/or antigen present in the dosage forms can be varied according to the nature of the anti-IgM agent, high affinity IL-2 receptor agonist, immunosuppressant (e.g., synthetic nanocarriers comprising an immunosuppressant) and/or antigen, the therapeutic benefit to be accomplished, and other such parameters. In embodiments, dose ranging studies can be conducted to establish optimal therapeutic amount of the anti-IgM agent, high affinity IL-2 receptor agonist, immunosuppressant (e.g., synthetic nanocarriers comprising an immunosuppressant) and/or antigen to be present in dosage forms. In embodiments, the anti-IgM agent, high affinity IL-2 receptor agonist, immunosuppressant (e.g., synthetic nanocarriers comprising an immunosuppressant) and/or antigen are present in dosage forms in an amount effective to generate a tolerogenic immune response to the antigen upon administration to a subject, such as according to the methods provided herein. In preferable embodiments, the anti-IgM agent, high affinity IL-2 receptor agonist, immunosuppressant (e.g., synthetic nanocarriers comprising an immunosuppressant) and/or antigen are present in dosage forms in an amount effective to suppress IgG production and/or enhance the production or development of regulatory T cells, such as CD4+ regulatory T cells, such as when concomitantly administered to a subject as provided herein. It may be possible to determine amounts of the anti-IgM agent, high affinity IL-2 receptor agonist, immunosuppressant (e.g., synthetic nanocarriers comprising an immunosuppressant) and/or antigen effective to generate desired immune responses using conventional dose ranging studies and techniques in subjects. Dosage forms may be administered at a variety of frequencies. In further embodiments, the anti-IgM agent, high affinity IL-2 receptor agonist, immunosuppressant (e.g., synthetic nanocarriers comprising an immunosuppressant) and/or antigen are present in dosage forms in an amount effective to reduce the production of IgG and/or enhance the production or development of regulatory T cells, such as CD4+ regulatory T cells.

Another aspect of the disclosure relates to kits. In some embodiments, the kit comprises immunosuppressant (e.g., synthetic nanocarriers comprising an immunosuppressant), an anti-IgM agent, and a high affinity IL-2 receptor agonist. In some embodiments the kit also comprises an antigen. The antigen may be attached to the synthetic nanocarriers comprising an immunosuppressant or other synthetic nanocarriers, in some embodiments. The synthetic nanocarriers, anti-IgM agent, high affinity IL-2 receptor agonist and any other components can be contained within separate containers in the kit. In some embodiments, the container is a vial or an ampoule. In some embodiments, the synthetic nanocarriers, anti-IgM agent, high affinity IL-2 receptor agonist and any other components are contained within a solution separate from the container, such that the synthetic nanocarriers, anti-IgM agent, high affinity IL-2 receptor agonist and any other components may be added to the container at a subsequent time. In preferred embodiments, synthetic nanocarriers, anti-IgM agent, high affinity IL-2 receptor agonist and any other components are not co-formulated with each other prior to administration. In some embodiments, the synthetic nanocarriers, anti-IgM agent, high affinity IL-2 receptor agonist and any other components are in lyophilized form each in a separate container, such that they may be reconstituted at a subsequent time. In some embodiments, the kit further comprises instructions for reconstitution, mixing, administration, etc. In some embodiments, the instructions include a description of the methods described herein. Instructions can be in any suitable form, e.g., as a printed insert or a label. In some embodiments, the kit further comprises one or more syringes or other means for administering the synthetic nanocarriers, anti-IgM agent, high affinity IL-2 receptor agonist and any other components.

### EXAMPLES

### Example 1: ImmTOR, αBAFF, and IL-2 Mutein Combination

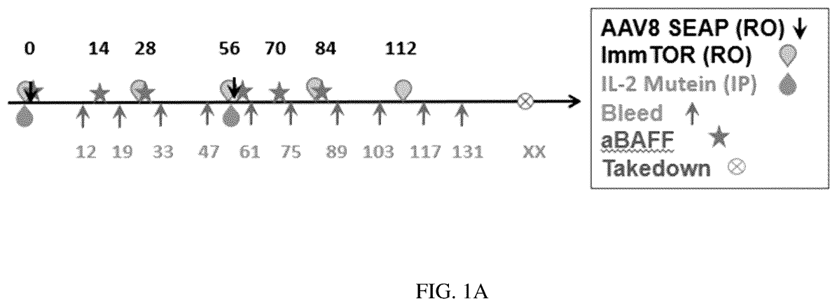

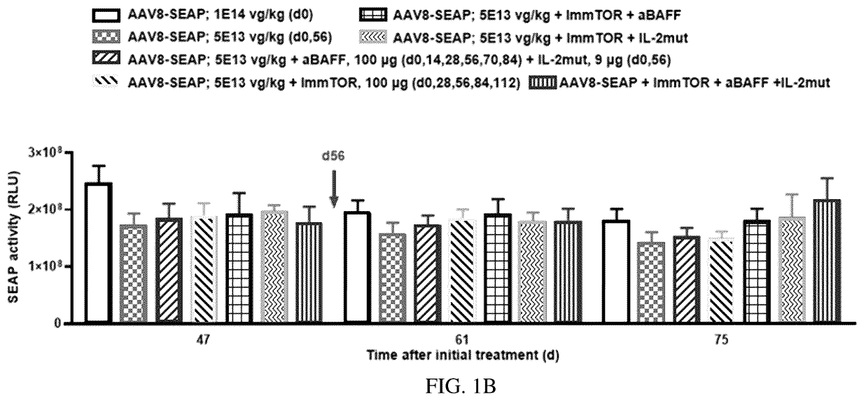

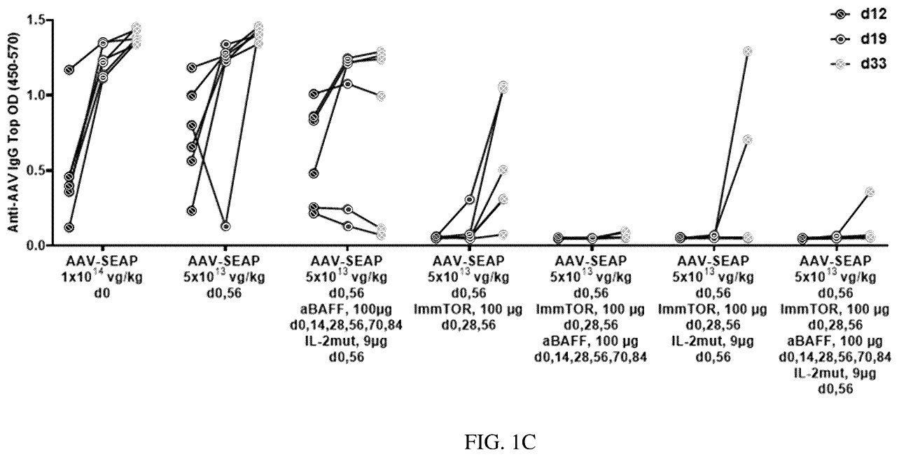

Mice were used to evaluate the effect of injecting AAV8-SEAP, αBAFF, and IL-2 mutein on IgG suppression and secreted embryonic alkaline phosphatase (SEAP) dynamics. Animals were distributed across 7 groups (TABLE 1 and FIG. 1A). Animals were evaluated (FIG. 1B). Mice treated with the combination of ImmTOR, αBAFF, and IL2-mutein showed significant SEAP elevation. These results indicate that the triple combination of ImmTOR, αBAFF, and IL-2 mutein can provide for increased AAV8-SEAP activity in in vivo. Animals were also evaluated for IgG suppression (FIG. 1C). IgG suppression was observed with administration of ImmTOR, αBAFF, and IL-2 mutein.

TABLE 1
Dosing regime for ImmTOR, IL-2 mutein, and αBAFF injections in mouse subjects

| Gr # | Prime d 0 | Boost d 56 | ImmTOR | aBAFF | IL-2mut | Aim or question asked |
|---|---|---|---|---|---|---|
| 1 | AAV8-SEAP, | None | None | None | None | ~1E14 vg/kg single high dose |
| | 1E14 vg/kg | | | | | baseline |
| | | | | | | transgene expression and |
| | | | | | | antibody induction |
| 2 | AAV8-SEAP, | AAV8-SEAP, | None | None | None | Repeat dosing of ~5E13 |
| | 5E13 vg/kg | 5E13 vg/ kg | | | | vg/kg |
| 3 | AAV8-SEAP | AAV8-SEAP | None | 100 μg d 0, | 9 μg d 0, | Repeat dosing of ~5E13 |
| | 5E13 vg/kg | 5E13 vg/kg | | 14, 28, 56, | 56 | vg/kg + IL-2 Mutein + 3 |
| | | | | 70, 84 | | biweekly aBAFF at/after |
| | | | | | | every AAV injection |
| 4 | AAV8-SEAP | AAV8-SEAP | 100 μg, d 0, | None | None | Repeat dosing of ~5E13 |
| | 5E13 vg/kg+ | 5E13 vg/kg + | 28, 56, 84, | | | vg/kg + ImmTOR + monthly |
| | ImmTOR | ImmTOR | 112 | | | ImmTOR |
| 5 | AAV8-SEAP | AAV8-SEAP | 100 μg, d 0, | 100 μg d 0, | None | Repeat dosing of ~5E13 |
| | 5E13 vg/kg + | 5E13 vg/kg + | 28, 56, 84, | 14, 28, 56, | | vg/kg + ImmTOR + monthly |
| | ImmTOR + | ImmTOR + | 112 | 70, 84 | | ImmTOR + 3 biweekly |
| | aBAFF | aBAFF | | | | aBAFF |
| 6 | AAV8-SEAP | AAV8-SEAP | 100 μg, d 0, | None | 9 μg d 0, | Repeat dosing of ~5E13 |
| | 5E13 vg/kg + | 5E13 vg/kg + | 28, 56, | | 56 | vg/kg + ImmTOR + IL-2 |
| | ImmTOR + | ImmTOR + | 84, 112 | | | mutein + monthly ImmTOR |
| | IL-2mut | IL-2mut | | | | |
| 7 | AAV8-SEAP | AAV8-SEAP | 100 μg, d 0, | 100 μg d 0, | 9 μg d 0, | Repeat dosing of ~5E13 |
| | 5E13 vg/kg + | 5E13 vg/kg + | 28, 56, 84, | 14, 28, 56, | 56 | vg/kg + ImmTOR + IL- |
| | ImmTOR + | ImmTOR + | 112 | 70, 84 | | 2mut + monthly ImmTOR + |
| | aBAFF + | aBAFF + | | | | 3 biweekly aBAFF |
| | IL-2mut | IL-2mut | | | | |

1. A composition comprising:
(a) an immunosuppressant,
(b) a high affinity IL-2 receptor agonist,
(c) an anti-IGM agent and,
(d) optionally, an antigen.

2. (canceled)

3. The composition of claim 1, wherein the immunosuppressant is comprised in synthetic nanocarriers and wherein the antigen is encapsulated in the synthetic nanocarriers.

4. A dosage form comprising the composition of claim 1.

5. A method comprising administering to a subject in need thereof:
(a) an immunosuppressant,
(b) a high affinity IL-2 receptor agonist,
(c) an anti-IGM agent, and,
(d) optionally, an antigen.

6. The method of claim 5, wherein the immunosuppressant, the high affinity IL-2 receptor agonist, the anti-IgM agent and, optionally, an antigen are administered concomitantly.

7. The method of claim 5, wherein (a), (b), (c) and, optionally, (d) are administered in an amount effective to enhance regulatory T cells (e.g., CD4+), such as antigen-specific regulatory T cells (e.g, CD4+).

8. The method of claim 5, wherein the subject has or is at risk of having an inflammatory disease, an autoimmune disease, an allergy, graft versus host disease, an undesired immune response against an antigen that is being administered or will be administered to the subject, or an undesired immune response against an antigen to which the subject is exposed or will be exposed.

9. (canceled)

10. The composition of claim 1, wherein the composition comprises the antigen and wherein the antigen is a therapeutic macromolecule.

11. (canceled)

12. The composition of claim 1, wherein the immunosuppressant comprises a statin, an mTOR inhibitor, a TGF-β signaling agent, a corticosteroid, an inhibitor of mitochondrial function, a P38 inhibitor, an NF-κB inhibitor, an adenosine receptor agonist, a prostaglandin E2 agonist, a phosphodiesterase 4 inhibitor, an HDAC inhibitor or a proteasome inhibitor.

13. (canceled)

14. The composition of claim 1, wherein the immunosuppressant is comprised in synthetic nanocarriers and the synthetic nanocarriers comprise lipid nanoparticles, polymeric nanoparticles, metallic nanoparticles, surfactant-based emulsions, dendrimers, buckyballs, nanowires, virus-like particles or peptide or protein particles.

15. The method or composition of claim 14, wherein the synthetic nanocarriers comprise polymeric nanoparticles.

16.-19. (canceled)

20. The composition of claim 14, wherein the mean of a particle size distribution obtained using dynamic light scattering of the synthetic nanocarriers is a diameter greater than 100 nm.

21.-32. (canceled)

33. The composition of claim 14, wherein an aspect ratio of the synthetic nanocarriers is greater than or equal to 1:1, 1:1.2, 1:1.5, 1:2, 1:3, 1:5, 1:7 or 1:10.

34. The composition of claim 14, wherein the load of immunosuppressant comprised in the synthetic nanocarriers, on average across the synthetic nanocarriers, is between 1% and 40% (weight/weight).

35.-45. (canceled)

46. The composition of claim 1, wherein the high affinity IL-2 receptor agonist is wild type IL-2, an IL-2 mutein, or an IL-2 fusion protein.

47. The composition of claim 1, wherein the anti-IgM agent is a/an IgM antagonist antibody, IgM antigen-binding fragment, IL-21 modulating agent, tyrosine kinase inhibitor, PI3K inhibitor, PKC inhibitor, APRIL antagonist, mizoribine, tofacitinib, or tetracyclines.

48.-51. (canceled)

52. The composition of claim 10, wherein the therapeutic macromolecule is a viral vector.

53.-56. (canceled)

57. The method of claim 5, wherein the antigen is a viral vector and wherein a dose of the viral vector is a lower dose vector and wherein the lower dose of the viral vector is less than 1e14 vector genomes/kg.

58. The method of claim 57, wherein when the dosing(s) comprise more than one dose of a viral vector, such as multiple lower doses of the viral vector, the doses of each dosing are administered over a 1 to 2 week period.

59. (canceled)

60. The method of claim 5, wherein the subject is experiencing or has experienced loss of transgene expression.

61. (canceled)
